(19) United States
(12) Patent Application Publication
(10) Pub. No.: US 2006/0223846 A1
(43) Pub. Date: Oct. 5, 2006

(54) Aza-bridged-bicyclic amino acid derivatives as alpha4 integrin antagonists

(76) Inventors: Alexey B. Dyatkin, Ambler, PA (US); Yong Gong, Warrington, PA (US); Wei He, Audubon, PA (US); Tamara Miskowski, Chalfont, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/370,665

(22) Filed: Mar. 8, 2006

Related U.S. Application Data
(60) Provisional application No. 60/659,710, filed on Mar. 8, 2005.

(51) Int. Cl. C07D 453/02; A61K 31/46

(52) U.S. Cl. 514/304; 546/125

(57) ABSTRACT

The invention is directed to aza-bridged-bicyclic compounds having Formula (I): 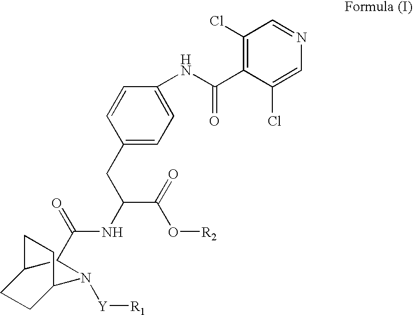 and pharmaceutically acceptable salts thereof. The compounds are useful α4 integrin receptor antagonists and, in particular, α4β1 and α4β7 integrin receptor antagonists. The invention is further directed to methods for use of the instant compounds for treating integrin mediated disorders including, but not limited to, inflammatory disorders, autoimmune disorders and cell-proliferative disorders, methods for preparing the compounds and methods for preparing the intermediates, derivatives and pharmaceutical compositions thereof.

This patent application claims benefit of U.S. Patent Application Ser. No. 60/659,710 filed on Mar. 8, 2005 entitled “AZA-BRIDGED BICYCLIC AMINO ACID DERIVATIVES AS α4 INTEGRIN ANTAGONISTS,” which is hereby incorporated by reference. This invention relates to novel compounds and methods for use in treating integrin mediated disorders. More particularly, this invention relates to novel derivatives of aza-bridged-bicyclic amino acid compounds useful as α4 integrin receptor antagonists, methods for treating integrin mediated disorders including, but not limited to, inflammatory, autoimmune and cell-proliferative disorders, methods for preparing the compounds and methods for preparing the intermediates, derivatives and pharmaceutical compositions thereof.

### FIELD OF THE INVENTION

### Background of the Invention

Integrin receptors are transmembrane, non-covalently linked heterodimers consisting of one α-chain and one β-chain. In addition to performing a structural adhesive function, integrin receptors transmit extracellular signals across the plasma membrane. The integrin receptor α₄β₁ (also referred to as VLA-4) mediates cell adhesion by binding with either of two protein ligands: vascular cell adhesion molecule-1 (VCAM-1) (Osborn, L.; et al., Cell, 1989, 59, 1203), or the alternatively-spliced fibronectin variant containing the type III connecting segment (CS-1) (Wayner, E. A.; et al., Cell Biol, 1989, 109, 1321). In contrast to the prototypical integrin receptors α5β1, GPIIb/IIIa and αᵥβ₃ that recognize the Arg-Gly-Asp (RGD) tripeptide sequence in their respective ligands, α₄β₁ binds to other primary protein sequences. The α₄β₁ integrin receptor recognizes Gln-Ile-Asp-Ser (QIDS) in VCAM-1 and Ile-Leu-Asp-Val (ILDV) in fibronectin. Although these sequences share a conserved Asp residue with RGD, they are otherwise unrelated. Additionally, recent studies have found that α4β1 binds the matrix ligand osteopontin (Bayless, K. J.; et al., J. Cell Sci., 1998, 111, 1165). The osteopontin ligand interaction with the α₄β₁ receptor may be very important as osteopontin is strongly up-regulated in inflammatory settings, including the inflamed lung.

The α₄β₁ integrin receptor is expressed at high levels on mast cells, mononuclear leukocytes, eosinophils, macrophages, and basophils (Adams, S. P.; et al., Ann. Rep. Med. Chem., 1999, 34, 179). The binding of α₄β₁ to cytokine-induced VCAM-1 on high-endothelial venules at sites of inflammation results in leukocyte/endothelium adhesion followed by extravasation into the inflamed tissue (Chuluyan, H. E.; et al., Springer Semin. Immunopathol., 1995, 16, 391). The role of mast cells and eosinophils in lung inflammation is well-established. Induction of VCAM-1 expression on airway endothelial cells seems to play a central role in lung inflammation. The α4β1 receptor interaction with VCAM-1 also exerts an important effect in stem cell adhesion to bone marrow stromal cells (Simmons, P. J.; et al., Blood, 1992, 80, 388).

The α₄β₇ integrin is expressed at high levels on lymphocytes and T cells. The trafficking of lymphocytes from the vasculature to normal mucosa and lymphoid tissues is mediated by adhesion of mucosal addressing cell adhesion molecule-1 (MAdCAM-1) with the integrin receptor α₄β₇. In an inflammatory setting, MAdCAM-1, an immunoglobulin superfamily adhesion molecule, specifically binds α4β7-expressing lymphocytes and participates in the homing of these cells to the mucosal endothelium. Cloning studies of human MAdCAM-1 have shown that the Leu-Asp-Thr-Ser-Leu (LDTSL) sequence of the CD loop is conserved. In fact, LDT-based peptides bind to α₄β₇ in a MAdCAM-1/RPMI-8866 cell adhesion assay with IC₅₀ values in the 1-10 uM range (Shroff, H. N.; et al., Bioorg. Med. Chem. Lett., 1998, 8, 1601).

The extensive biology mediated by integrins in general and compelling data for the pathophysiological role of the leukocyte cell adhesion receptor α4β1 have spurred interest in the study of α4β1 antagonists in vivo. Cellular adhesion and migration mediated through the β1 integrins are critical components of cellular recruitment processes. The integrin α4β1 provides a key co-stimulatory signal supporting cell activation leading to growth factor and cytokine production and mediator release. Through recognition of the extracellular matrix, α4β1 increases the survival of activated cells by inhibiting apoptosis (Yoshikawa, H.; et al., J. Immunol., 1996, 156, 1832).

Monoclonal antibodies directed against α4β1 or VCAM-1 have been shown to be effective modulators in animal models of chronic inflammatory diseases such as asthma (Laberge, S.; et al., Am. J. Respir. Crit. Care Med., 1995, 151, 822), rheumatoid arthritis (Barbadillo, C.; et al., Springer Semin. Immunopathol., 1995, 16, 375) and inflammatory bowel disease (Powrie, F.; et al., Ther. Immunol., 1995, 2, 115). The initial research in the low molecular weight α4β1 antagonist arena has focused on simple linear analogues of the prototype Leu-Asp-Val sequence. Phenylacetyl-Leu-Asp-Phe-D-Pro-NH2 (having an α4β1 IC₅₀ value of 2 uM) exhibited efficacy similar to the α4 antibody PS/2 in a mouse model of oxazolone-induced contact hypersensitivity when administered at 6 mg/kg, sc (Tamraz, S.; et al., Springer Semin. Immunopathol. 1995, 16, 437). This tetrapeptide was also effective in a hyperlipidemic rabbit heterotopic heart allograft model (Molossi, S.; et al., J. Clin. Invest. 1995, 95, 2601).

Animal models of asthma have shown that the peptide antagonist BIO-1211 inhibits eosinophilia and airway hyperresponsiveness (Lin, K-C.; et al., J. Med. Chem. 1999, 42, 920). Pre-treatment of allergic sheep with a 3 mg nebulized dose of BIO-1211 (having an α4β1 IC₅₀ value of 1 nM; 1000-fold selective over α4β7) inhibited early and late airway responses following antigen challenge and prevented development of nonspecific airway hyperresponsiveness to carbachol. These results suggest that compounds like BIO-1211 can effect broad pleiotropic activities by acting at α4β1 to achieve pronounced efficacy similar to corticosteroids.

VLA-4 antagonism may also be effective in reducing restenosis following percutaneous coronary interventions. Administration of an anti-α4 antibody attenuated smooth muscle cell migration associated with electrical injury of rabbit carotid arteries (Kling D, Fingerle J, Harlan J M, Lobb, R R and Lang, F, Mononuclear leukocytes invade rabbit arterial intima during thickening formation via CD-18 and VLA-4-dependent mechanisms and stimulate smooth muscle migration, Circ. Res., 1995, 77, 1121-1128) and was shown to reduce neointimal formation in baboon carotid arteries following endarterectomy (Lumsden A B, Chen C, Hughes J D, Kelly A B, Hanson S and Harker L, Anti-VLA-4 antibody reduces intimal hyperplasia in the endarterectomized carotid artery in non-human primates, J. Vasc. Surg., 1997, 26, 87-93). Furthermore, treatment with z anti-α4 antibody was associated with less neoadventitia formation and less lumenal narrowing 14 days after balloon injury of porcine coronary arteries (Labinez M, Hoffert C, pels K, Aggarawal S, Pepinsky R B, Leonw D, Koteliansky V, Lobb, R R and O'Brien E O, Infusion on and anti-alpha4 integrin antibody is associated with less adventitial formation after balloon injury of porcine coronary arteries, Can. J. Cardiol., 2000, 16, 187-196).

The recruitment of leukocytes, particularly monocytes to the vessel wall is a key component in the development of atherosclerotic lesions. VCAM-1 expression has been reported on endothelial cells in atherosclerotic lesions in humans (O'Brien K D, Allen M D, McDonald T O, Chait A, Harlan J M, Fishbein D, McCarty J, Ferguson M, Hudkins K, Benjamin C D, et al., Vascular cell adhesion molecule-1 is expressed in human atherosclerotic plaques: implications for the mode of progression of advanced atherosclerosis, J. Clin. Invest., 1993, 92, 945-951), mice (Nakahima Y, Raines E W, Plump A S, Breslow J L and Ross R, Upregulation of VCAM-1 and ICAM-1 at atherosclerotic-prone sites on the endothelium of ApoE-deficient mouse, Arterioscler. Thromb. Vasc. Biol., 1998, 18, 842-851) and rabbits (Ilyama K, Hajra L, Iiyam M, Li, H, DiChura M, Medoff B D and Cybulsky M I, Patterns of vascular cell adhesion molecule-1 and intercellular adhesion molecule-1 expression in rabbit and mouse atherosclerotic lesion and at sites predisposed to lesion formation, Circ. Res., 1999, 85, 199-207). Furthermore, a synthetic peptidomimetic of the connecting segment-1 (CS-1) which blocks α₄β₁ on the leukocyte demonstrated reduced leukocyte homing and lipid accumulation in the aortic sinus in both wild type mice and mice with allow density lipoprotein null mutation (LDLR −/−) maintained on a high fat diet (Shih P T, Brennan M L, Vora D K, Territo M C, Strahl D, Elices M J, Aldons J and Berliner J A, Blocking very late antigen-4 integrin decreases leukocyte entry and fatty streak formation in mice fed an atherogenic diet, Circ. Res., 1999, 84, 345-351). In studies using isolated carotid arteries from ApoE −/− mice (these mice develop spontaneous arterial atherosclerotic lesions with advanced lesions similar to those observed in humans), administration on blocking antibodies to VCAM-1 inhibited the majority of adhesion of monocytes or U937 cells on early atherosclerotic endothelia. In addition, a peptide which inhibits binding of α4β1 to both VCAM-1 and fibronectin was also effective in this model (Huo Y, Hafez-Moghadem A and Ley K, Role of vascular cell adhesion molecule-1 and fibronectin connecting segment-1 in monocyte rolling and adhesion on early atherosclerotic lesions, Circ. Res., 2000, 87, 153-159). These data support the role of α₄β₁ in regulating leukocyte recruitment in early and advanced atherosclerotic lesions.

Antibodies to MAdCAM-1 or integrin α4β7 inhibit lymphocyte binding to affinity-purified MAdCAM-1 or MAdCAM-1 transfectants in vitro (Hamann, A.; et al., J. Immunol. 1994, 152, 3282). The antibodies also block localization of lymphocytes to Peyer's patches. Murine MAdCAM-1 recognizes only α4β7 positive human lymphocyte cells lines and α4β7-high memory T cells. An in vivo role of α4β7 in inflammation has been suggested by increased expression of MAdCAM-1 on HEV-type vessels in the chronically inflamed pancreas of the non-obese mouse (Hanninen, A. C.; et al., J. Clin. Invest. 1993, 92, 2509). In fact, animal models underscore a significant function of α4β7 in both colitis (Fong, S.; et al., Immunol. Res. 1997, 16, 299) and lymphocytic inflammation of pancreatic islets or development of diabetes (Yang, X.; et al., Diabetes 1997, 46, 1542).

Accordingly, it is an object of the present invention to provide aza-bridged-bicyclic compounds that are α4 integrin receptor antagonists; more particularly, the α₄β₁ and the α₄β₇ integrin receptor. It is also an object of the present invention to provide a process for preparing derivatives of aza-bridged-bicyclic amino acid compounds, compositions, intermediates and derivatives thereof. It is a further object of the invention to provide methods for the treatment of integrin mediated disorders that are ameliorated by inhibition of the α₄β₁ and α₄β₇ integrin receptor including, but not limited to, inflammatory, autoimmune and cell-proliferative disorders.

### SUMMARY OF THE INVENTION

An embodiment of the present invention is directed to aza-bridged-bicyclic compounds having Formula (I): 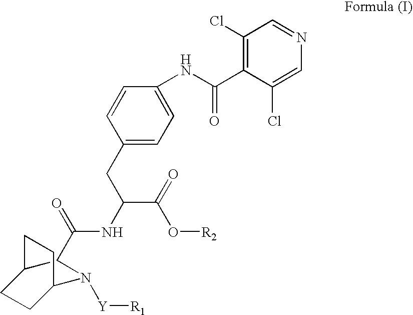
wherein
Y is selected from the group consisting of bond, —C(O)—, C(O)O— and C(O)NH;
R₁ is selected from the group consisting of R₃ and R₄;
R₂ is independently selected from the group consisting of hydrogen and C₁₋₈alkyl; wherein C₁₋₈alkyl is optionally substituted with one to three substituents independently selected from amino, N—(C₁₋₄alkyl)amino, N,N—(C₁₋₄dialkyl)amino, hydroxy, C₁₋₄alkoxy, —CF₃ and —OCF₃:
R₃ and R₅ are independently selected from the group consisting of cycloalkyl, heterocyclyl, aryl and heteroaryl optionally substituted with one to five substituents independently selected from the group consisting of halogen, C₁₋₆alkyl, C₂₋₆alkenyl, C₂₋₆alkynyl, C₁₋₆alkoxy, C₁₋₆alkylcarbonyl, C₁₋₄alkoxycarbonyl, carboxyl, aryl, heteroaryl, arylcarbonyl, heteroarylcarbonyl, arylsulfonyl, amino, N—(C₁₋₈alkyl)amino, N,N—(C₁₋₈dialkyl)amino, —CF₃ and —OCF₃; wherein cycloalkyl and heterocyclyl are optionally substituted with one to three oxo substituents; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N—(C₁₋₈alkyl)amino, N,N—(C₁₋₈dialkyl)amino, —CF₃ and —OCF₃;
R₄, is independently selected from the group consisting of C₁₋₆alkyl, C₂₋₆alkenyl, C₂₋₆alkynyl, and (halo)₁₋₃(C₁₋₆)alkyl; wherein C₁₋₈alkyl, C₂₋₈alkenyl and C₂₋₈alkynyl are optionally substituted on a terminal carbon with one to three substituents independently selected from R₅;
and pharmaceutically acceptable salts, racemic mixtures, diastereomers and enantiomers thereof.

An embodiment of the present invention is also directed to a process for preparing the instant aza-bridged-bicyclic compounds, compositions, intermediates and derivatives thereof. Another embodiment of the present invention is directed to pharmaceutical compositions comprising the compounds of the present invention.

The aza-bridged-bicyclic amino acid derivatives of the present invention are useful α4 integrin receptor antagonists and, more particularly, α₄β₁ and α₄β₇ integrin receptor antagonists. A further embodiment of the present invention is directed to a method for the treatment of integrin mediated disorders that are ameliorated by inhibition of the α₄β₁ and α₄β₇ integrin receptor including, but not limited to, inflammatory, autoimmune and cell-proliferative disorders. In an illustration of the invention, the inflammatory, autoimmune and cell-proliferative disorders include, but are not limited to, inflammation and autoimmunity, asthma and bronchoconstriction, restenosis and atherosclerosis, psoriasis, rheumatoid arthritis, inflammatory bowel disease, transplant rejection and multiple sclerosis.

### DETAILED DESCRIPTION OF THE INVENTION

We have discovered that the position and type of the substituents on the phenyl group of the phenylalanine amino acid, in combination with stereochemistry, have a significant effect on the α₄β₁ and α₄β₇ integrin receptor antagonist activity of the aza-bridged-bicyclic compounds of the present invention. Relative to the above generic description, certain compounds having Formula (I) are preferred.

In addition to the above discoveries relative to the structure of the compounds of the present invention, we have experimentally determined that the stereochemistry significantly affects the α₄β₁, and α₄β₇ integrin receptor antagonist activity of certain compounds. In addition to racemic mixtures demonstrating activity as α₄β₁ and α₄β₇ integrin receptor antagonists, experimental results have shown that individual diastereomers each have either a significantly increased or significantly decreased activity as an α₄β₁ and α₄β₇ integrin receptor antagonist.

Although the racemic mixtures have significant activity compared to the resolved diastereomers, the (S,S) diastereomers appear to generally have higher activity than the (R,S) diastereomers. The scope of the present invention is intended to encompass all racemic mixtures, enantiomers and diastereomers including, but not limited to, (R/S,S), (R/S,R), (S,R/S), (R,R/S), (S,S), (R,S), (S,R) and (R,R) diastereomers and enantiomers of the compounds of the present invention without limitation.

Preferred embodiments include those compounds wherein R₁ is selected from R₃. R₃ is preferably selected from the group consisting of cycloalkyl aryl and heteroaryl optionally substituted with one to five substituents independently selected from the group consisting of halogen, C₁₋₆alkyl, C₂₋₈alkenyl, C₂₋₈alkynyl, C₁₋₈alkoxy, C₁₋₈alkylcarbonyl, C₁₋₈alkoxycarbonyl, carboxyl, aryl, heteroaryl, arylcarbonyl, heteroarylcarbonyl, arylsulfonyl, amino, N—(C₁₋₈alkyl)amino, N,N—(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₈alkyl, C₂₋₈alkenyl, C₂₋₈alkynyl, C₁₋₈alkoxy, carboxyl, amino, N—(C₁₋₈alkyl)amino, N,N—(C₁₋₈dialkyl)amino, —CF₃ and —OCF₃.

In another preferred embodiment R₃ is selected from the group consisting of cycloalkyl, aryl and heteroaryl optionally substituted with one to five substituents independently selected from the group consisting of halogen, C₁₋₆alkyl, C₂₋₆alkenyl, C₂₋₆alkynyl, C₁₋₆alkoxy, C₁₋₈alkylcarbonyl, C₁₋₆alkoxycarbonyl, carboxyl, aryl, heteroaryl, arylcarbonyl, heteroarylcarbonyl, arylsulfonyl, amino, N—(C₁₋₆alkyl)amino, N,N—(C₁₋₆dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₆alkyl, C₂₋₆alkenyl, C₂₋₆alkynyl, C₁₋₆alkoxy, carboxyl, amino, N—(C₁₋₆alkyl)amino, N,N—(C₁₋₆dialkyl)amino, —CF₃ and —OCF₃.

In another preferred embodiment R₃ is selected from the group consisting of aryl optionally substituted with one to five substituents independently selected from the group consisting of halogen, C₁₋₆alkyl, C₂₋₆alkenyl, C₂₋₆alkynyl, C₁₋₆alkoxy, C₁₋₆alkylcarbonyl, C₁₋₆alkoxycarbonyl, carboxyl, aryl, heteroaryl, arylcarbonyl, heteroarylcarbonyl, arylsulfonyl, amino, N—(C₁₋₈alkyl)amino, N,N—(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₆alkyl, C₂₋₆alkenyl, C₁₋₆alkoxy, carboxyl, amino, N—(C₁₋₆alkyl)amino, N,N—(C₁₋₆dialkyl)amino, —CF₃ and —OCF₃.

In another preferred embodiments include those compounds wherein R₃ is selected from the group consisting of tolyl, phenyl, 2-chlorophenyl, 3-chlorophenyl, 2-fluorophenyl, 3,5-dichlorophenyl, 2,5-dimethoxyphenyl, 4-fluoro-biphen-2-yl, 2-trifluoromethylphenyl and 4-fluoro-biphen-2-yl.

In another more preferred embodiment when Y is a bond 2,5-dimethyoxyphenyl, 2-fluorophenyl, 3,5-dichlorophenyl and 4-fluoro-biphenyl-2-yl. In a more preferred embodiment when Y is selected from the group consisting of —C(O)— and —C(O)O— R₃ is selected from the group consisting of 2,5-dimethoxyphenyl, 2-fluoropheyl, 3,5-dichlorophenyl and 4-fluoro-biphenyl-2-yl.

Preferred embodiments include those compounds wherein R₄ is selected from the group consisting of C₁₋₈alkyl and C₂₋₈alkynyl optionally substituted on a terminal carbon with R₅. Preferably, R₄ is selected from the group consisting of C₁₋₆alkyl and C₂₋₆alkynyl optionally substituted on a terminal carbon with R₅. More preferably, R₄ is selected from the group consisting of C₁₋₄alkyl and C₂₋₆alkynyl optionally substituted on a terminal carbon with R₅. Most preferably, R₄ is selected from the group consisting of methyl, ethyl, propyl, butyl, t-butyl and ethynyl; wherein the methyl, ethyl, propyl, butyl, and ethynyl are substituted on a terminal carbon with a substituent R₅.

In another preferred embodiment includes compounds where Y Is a bond and R₄ is C₁₋₄alkyl optionally substituted with R₅, and R₅ is selected from the group consisting of heterocyclyl and aryl optionally substituted with C₁₋₄alkyl, C₁₋₄alkoxy, N—(C₁₋₄alkyl)amino, N,N—(C₁₋₄dialkyl)amino, —CF₃ and —OCF₃

Preferably, R₄ is selected from the group consisting of 3,3-dimethyl-butyl, 5-chloro-2,3-dihydro-thophe-2-ylmethyl and 5-chloro-thiophen-2-ylmethyl.

In another preferred embodiment includes compounds where Y is —C(O)NH— and R₄ is C₁₋₄alkyl and R₅ phenyl optionally substituted with one to three substitutents selected from the group consisting of halogen, C₁₋₄alkyl, C₂-4alkenyl, C₁₋₄alkoxy, carboxyl, amino, N—(C₁₋₄alkyl)amino, N,N—(C₁₋₄dialkyl)amino, —CF₃ and —OCF₃. In a more preferred embodiment includes compounds where Y is —C(O)NH— and R₄ is C₁₋₄alkyl and R₅ phenyl.

Preferred embodiments include those compounds wherein Y is selected from the group consisting of —C(O)— and —C(O)O— and R₅ is preferably

cycloalkyl, heterocyclyl, aryl and heteroaryl optionally substituted with one to five substituents independently selected from the group consisting of halogen, C₁₋₆alkyl, C₁₋₆alkoxy, C₁₋₆alkylcarbonyl, C₁₋₆alkoxycarbonyl, carboxyl, amino, N—(C₁₋₆alkyl)amino, N,N—(C₁₋₆dialkyl)amino, —CF₃ and —OCF₃.

Another preferred embodiment includes compounds wherein Y is selected from the group consisting of —C(O)— and —C(O)O—, R₄ is methyl, and R₅ is selected from the group consisting of phenyl; 4-methylphenyl; 2-methoxyphenyl; 3-methoxyphenyl; 4-methoxyphenyl; 3,5-dimethoxyphenyl; 3,6-dimethoxyphenyl; 2,6-dichlorophenyl; 2-trifluoromethylphenyl; naphthalene-2-yl; thiophen-2-yl; thiophen-3-yl; pyridin-2-yl; pyridin-3-yl; pyridin-4-yl; 5-methyl-pyrazol-1-yl; tetrazol-1-; benzo[1,3]dioxol-5-yl; benzo[b]thiophen-3-yl; and tetrahydro-pyran-4-yl.

Another preferred embodiment includes compounds wherein Y is selected from the group consisting of —C(O)— and —C(O)O—, R₄ is ethyl and R₅ is selected from the group consisting of phenyl; 3-methoxyphenyl; 4-methoxyphenyl; 4-chlorophenyl; 2-fluorophenyl; 4-trifluoromethylphenyl; 3,5-ditrifluoromethylphenyl; thiophen-2-yl; 2-piperazin-1-yl; 2(4-tert-butoxycarbonyl-piperazin-1-yl); 2-piperidin-1-yl; fyran-3-yl; and 2-cyclopentyl.

Another preferred embodiment includes compounds wherein Y is selected from the group consisting of —C(O)— and —C(O)O—, R₄ is vinyl and R₅ is selected from the group consisting of 1-methyl-2-phenyl; and 2-(2-methoxy-phenyl).

Another preferred embodiment includes compounds wherein Y is selected from the group consisting of —C(O)— and —C(O)O—, R₄ is propyl, and R₅ is selected from the group consisting of -phenyl; 3-cyclohexyl; and 2,2-dimethyl.

Another preferred embodiment includes compound wherein Y is selected from the group consisting of —C(O)— and —C(O)O—, R₄ is butyl and R₅ is selected from the group consisting of 3 3,3-dimethyl; and 3-methyl.

Preferred embodiments include those compounds wherein R₂ is selected from the group consisting of hydrogen and C₁₋₄alkyl. More preferably, R₂ is selected from the group consisting of hydrogen and methyl.

Embodiments of the aza-bridged-bicyclic amino acid compounds of the present invention include those compounds of Formula (I) shown in Table I of the formula:

TABLE I

| | |
|---|---|
| | Formula (I) |
| 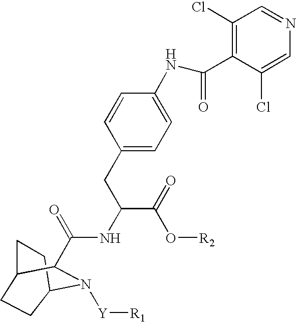 | |

wherein Y, and R₁ are dependently selected from the group consisting of:

| | | | * |
|---|---|---|---|
| Cpd | Y | R₁ | Config. |
| 1 | —C(O)— | 3-Methoxy-benzyl | R,S |
| 2 | —C(O)— | 2-Trifluoromethyl-benzyl | S |
| 3 | —C(O)— | Thiophen-3-ylmethyl | R,S |
| 4 | —C(O)— | Pyridin-2-ylmethyl | R,S |
| 5 | —C(O)— | Pyridin-3-ylmethyl | R,S |
| 6 | —C(O)— | 2,6-Dichloro-benzyl | R,S |
| 7 | —C(O)— | Naphthalen-2-ylmethyl | R,S |
| 8 | —C(O)— | 2,5-Dimethoxyphenyl | R,S |
| 9 | —C(O)— | 2-Fluorophenyl | R,S |
| 10 | —C(O)— | 3,5-Dichlorophenyl | R,S |
| 11 | —C(O)— | 4-Fluoro-biphenyl-2-yl | R,S |
| 12 | —C(O)— | 2-Trifluoromethylphenyl | R,S |
| 13 | —C(O)— | 4-Pentyl-bicyclo[2.2.2]oct-1-yl | R,S |
| 14 | —C(O)— | 2,6-Dichlorophenyl | R,S |
| 15 | —C(O)— | 5-Methyl-pyrazol-1-ylmethyl | R,S |
| 16 | bond | 3,3-Dimethyl-butyl | S |
| 17 | bond | 3-Phenyl-propyl | S |
| 18 | bond | Benzyl | S |
| 19 | bond | 5-Chloro-thiophen-2-ylmethyl | S |
| 20 | —C(O)— | Phenyl | S |
| | NH— | | |
| 21 | —C(O)— | 4-Tolyl | S |
| | NH— | | |
| 22 | —C(O)— | 2-Chlorophenyl | S |
| | NH— | | |
| 23 | —C(O)— | 3-Chlorophenyl | S |
| | NH— | | |
| 24 | —C(O)— | 4-Chlorophenyl | S |
| | NH— | | |
| 25 | —C(O)— | Benzyl | S |
| | NH— | | |
| 26 | —C(O)— | tert-Butyl | R,S |
| 27 | —C(O)— | 2,2-Dimethyl-propyl | R,S |
| 28 | —C(O)— | 2-Cyclopentyl-ethyl | R,S |
| 29 | —C(O)— | 2-Methoxy-benzyl | R,S |
| 30 | —C(O)— | 4-Methoxy-benzyl | R,S |
| 31 | —C(O)— | 3,5-Dimethoxy-benzyl | |
| 32 | —C(O)— | Thiophen-2-ylmethyl | R,S |
| 33 | —C(O)— | 3,6-Dimethoxy-benzyl | |
| 34 | —C(O)— | 2,2-Dimethyl-propyl | S |
| 35 | —C(O)— | 2-(4-tert-Butoxycarbonyl-piperazin-1-yl)- | S |
| | | ethyl | |
| 36 | —C(O)— | Benzo[b]thiophen-3-ylmethyl | R,S |
| 37 | —C(O)— | 3-Cyclohexyl-propyl | R,S |
| 38 | —C(O)— | 3-Methyl-butyl | R,S |
| 39 | —C(O)— | Phenethyl | R,S |
| 40 | —C(O)— | 1-Methyl-2-phenyl-vinyl | S |
| 41 | —C(O)— | Tetrahydro-pyran-4-ylmethyl | S |
| 42 | —C(O)— | Benzyl | S |
| | O— | | |
| 43 | —C(O)— | 2-Piperidin-1-yl-ethyl | R,S |
| 44 | —C(O)— | Pyridin-4-ylmethyl- | R,S |
| 45 | —C(O)— | 2-(2-Methoxy-phenyl)-vinyl | S |
| 46 | —C(O)— | 2-Benzo[1,3]dioxol-5-yl-vinyl | R,S |
| 47 | —C(O)— | Thiophen-2-yl-ethyl | R,S |
| 48 | —C(O)— | 4-Methoxyphenethyl | R,S |
| 49 | —C(O)— | 3-Methoxyphenethyl | R,S |
| 50 | —C(O)— | 4-Trifluoromethylphenethyl | R,S |
| 51 | —C(O)— | 4-Chlorophenethyl | R,S |
| 52 | —C(O)— | 2-Fluorophenethyl | R,S |
| 53 | —C(O)— | 2-Piperazin-1-yl-ethyl | R,S |
| 54 | —C(O)— | Furan-3-yl-ethyl | S |
| 55 | —C(O)— | 3,5-Ditrifluoromethyl-phenethyl | S |
| 56 | —C(O)— | 4-Dimethylamino-benzyl | S |
| 57 | —C(O)— | Phenylethynyl | R,S |
| 58 | bond | (4-MeC₆H₄)CH₂CH₂— | R,S |
| 59 | —CO— | 3-Methoxybenzyl | R,S |
| 60 | —CO— | Thiophen-3-ylmethyl | R,S |
| 61 | —CO— | 3-Phenoxylbenzyl | R,S |
| 62 | —CO— | 3-Benzo[b]thiophenylmethyl | R,S |
| 63 | —CO— | 2-Phenoxyphenyl | R,S |
| 64 | —CO— | Benzyloxy | R,S |
| 65 | —CO— | Pyridin-3-ylethyl | R,S |
| 66 | —CO— | 2-2-Methoxyphenethyl | R,S |

The compounds of the present invention may also be present in the form of pharmaceutically acceptable salts. For use in medicine, the salts of the compounds of this invention refer to non-toxic “pharmaceutically acceptable salts” (Ref. International J. Pharm., 1986, 33, 201-217; J. Pharm. Sci., 1997 (January), 66, 1, 1). Other salts may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds, which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. Where the processes for the preparation of the compounds according to the invention give rise to mixtures of stereoisomers, these isomers may be separated by conventional techniques such as preparative chromatography. The compounds may be prepared in racemic form or as individual enantiomers or diasteromers by either stereospecific synthesis or by resolution. The compounds may, for example, be resolved into their component enantiomers or diasteromers by standard techniques, such as the formation of stereoisomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of stereoisomeric esters or amides, followed by chromatographic separation and removal of the chiral auxiliary. Alternatively, the compounds may be resolved using a chiral HPLC column. It is to be understood that all stereoisomers, racemic mixtures, diastereomers and enantiomers thereof are encompassed within the scope of the present invention.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

As used herein, unless otherwise noted, “alkyl” and “alkoxy” whether used alone or as part of a substituent group refers to straight and branched carbon chains having 1 to 8 carbon atoms or any number within this range. Similarly, alkenyl and alkynyl groups include straight and branched chain alkenes and alkynes having 2 to 8 carbon atoms or any number within this range, wherein an alkenyl chain has at least one double bond in the chain and an alkynyl chain has at least one triple bond in the chain. Alkoxy radicals are oxygen ethers formed from the previously described straight or branched chain alkyl groups.

As used herein, unless otherwise noted “oxo” whether used alone or as part of a substituent group refers to an O═ to either a carbon or a sulfur atom. For example, phthalimide and saccharin are examples of compounds with oxo substituents.

The term “cycloalkyl,” as used herein, refers to an optionally substituted, stable, saturated or partially saturated monocyclic or bicyclic ring system containing from 3 to 8 ring carbons and preferably 5 to 7 ring carbons. Examples of such cyclic alkyl rings include cyclopropyl, cyclobutyl, cyclopentyl, cyclohexyl or cycloheptyl.

The term “heterocyclyl” as used herein refers to an optionally substituted, stable, saturated or partially saturated 5 or 6 membered monocyclic or bicyclic ring systems which consists of carbon atoms and from one to three heteroatoms selected from N, O or S; a saturated or partially unsaturated 5-6 membered heterocylic ring as previously defined fused to a heteroaryl as hereinafter defined; or a saturated, partially unsaturated or benzofused 5 to 6 membered heterocylic ring as previously defined. Examples of heterocyclyl groups include, but are not limited to, pyrrolinyl (including 2H-pyrrole, 2-pyrrolinyl or 3-pyrrolinyl), pyrrolidinyl, dioxolanyl, 2-imidazolinyl, imidazolidinyl, 2-pyrazolinyl, pyrazolidinyl, piperidinyl, dioxanyl, morpholinyl, thiomorpholinyl, piperazinyl or 2-benzo[1,3]dioxolyl. The heterocyclyl group may be attached at any heteroatom or carbon atom, which results in the creation of a stable structure.

The term “aryl”, as used herein, refers to optionally substituted aromatic groups comprising a stable six membered monocyclic or ten membered bicyclic aromatic ring system, which consists of carbon atoms. Examples of aryl groups include, but are not limited to, phenyl or naphthalenyl.

The term “heteroaryl” as used herein represents a stable five or six membered monocyclic aromatic ring system or a nine or ten membered benzo-fused heteroaromatic ring system which consists of carbon atoms and from one to three heteroatoms selected from N, O or S. The heteroaryl group may be attached at any heteroatom or carbon atom, which results in the creation of a stable structure.

The term “arylalkyl” means an alkyl group substituted with an aryl group (e.g., benzyl, phenethyl). The term “arylalkoxy” indicates an alkoxy group substituted with an aryl group (e.g., benzyloxy, phenethoxy, etc.). Similarly, the term “aryloxy” indicates an oxy group substituted with an aryl group (e.g., phenoxy).

Whenever the term “alkyl” or “aryl” or either of their prefix roots appear in a name of a substituent (e.g., aralkyl, alkylamino) it shall be interpreted as including those limitations given above for “alkyl” and “aryl.” Designated numbers of carbon atoms (e.g., C₁₋₆) shall refer independently to the number of carbon atoms in an alkyl or cycloalkyl moiety or to the alkyl portion of a larger substituent in which alkyl appears as its prefix root.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

The aza-bridged-bicyclic amino acid compounds of the present invention are useful α4 integrin receptor antagonists and, more particularly, α4β1 and α4β7 integrin receptor antagonists for treating a variety of integrin mediated disorders that are ameliorated by inhibition of the α4β1 and α4β7 integrin receptor including, but not limited to, inflammatory, autoimmune and cell-proliferative disorders.

Illustrative of the invention is a pharmaceutical composition comprising a pharmaceutically acceptable carrier and any of the compounds described above. Also illustrative of the invention is a pharmaceutical composition made by mixing any of the compounds described above and a pharmaceutically acceptable carrier. A further illustration of the invention is a process for making a pharmaceutical composition comprising mixing any of the compounds described above and a pharmaceutically acceptable carrier. The present invention also provides pharmaceutical compositions comprising one or more compounds of this invention in association with a pharmaceutically acceptable carrier.

An example of the invention is a method for the treatment of integrin mediated disorders in a subject in need thereof comprising administering to the subject a therapeutically effective amount of any of the compounds or pharmaceutical compositions described above. Also included in the invention is the use of a compound of Formula (I) for the preparation of a medicament for treating an integrin mediated disorder in a subject in need thereof.

Further exemplifying the invention is the method for the treatment of integrin mediated disorders, wherein the therapeutically effective amount of the compound is from about 0.01 mg/kg/day to about 30 mg/kg/day.

In accordance with the methods of the present invention, the individual components of the pharmaceutical compositions described herein can be administered separately at different times during the course of therapy or concurrently in divided or single combination forms. The instant invention is therefore to be understood as embracing all such regimes of simultaneous or alternating treatment and the term “administering” is to be interpreted accordingly.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human, that is being sought by a researcher, veterinarian, medical doctor, or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

The utility of the compounds to treat integrin mediated disorders can be determined according to the procedures herein. The present invention therefore provides a method for the treatment of integrin mediated disorders in a subject in need thereof which comprises administering any of the compounds as defined herein in a quantity effective to inhibit the α4β1 and α4β7 integrin receptor including, but not limited to, inflammatory, autoimmune and cell-proliferative disorders. Accordingly, a compound of the present invention may be administered by any conventional route of administration including, but not limited to oral, nasal, pulmonary, sublingual, ocular, transdermal, rectal, vaginal and parenteral (i.e. subcutaneous, intramuscular, intradermal, intravenous etc.).

To prepare the pharmaceutical compositions of this invention, one or more compounds of Formula (I) or salt thereof as the active ingredient, is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing a pharmaceutical composition of the present invention in liquid dosage form for oral, topical and parenteral administration, any of the usual pharmaceutical media or excipients may be employed. Thus, for liquid dosage forms, such as suspensions (i.e. colloids, emulsions and dispersions) and solutions, suitable carriers and additives include but are not limited to pharmaceutically acceptable wetting agents, dispersants, flocculation agents, thickeners, pH control agents (i.e. buffers), osmotic agents, coloring agents, flavors, fragrances, preservatives (i.e. to control microbial growth, etc.) and a liquid vehicle may be employed. Not all of the components listed above will be required for each liquid dosage form.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg/kg to about 300 mg/kg (preferably from about 0.01 mg/kg to about 100 mg/kg; and, more preferably, from about 0.01 mg/kg to about 30 mg/kg) and may be given at a dosage of from about 0.01 mg/kg/day to about 300 mg/kg/day (preferably from about 0.01 mg/kg/day to about 100 mg/kg/day and more preferably from about 0.01 mg/kg/day to about 30 mg/kg/day). Preferably, the method for the treatment of integrin mediated disorders described in the present invention using any of the compounds as defined herein, the dosage form will contain a pharmaceutically acceptable carrier containing between from about 0.01 mg to about 100 mg; and, more preferably, from about 5 mg to about 50 mg of the compound, and may be constituted into any form suitable for the mode of administration selected. The dosages, however, may be varied depending upon the requirement of the subjects, the severity of the condition being treated and the compound being employed. The use of either daily administration or post-periodic dosing may be employed.

Preferably these compositions are in unit dosage forms from such as tablets, pills, capsules, dry powders for reconstitution or inhalation, granules, lozenges, sterile parenteral solutions or suspensions, metered aerosol or liquid sprays, drops, ampoules, autoinjector devices or suppositories for administration by oral, intranasal, sublingual, intraocular, transdermal, parenteral, rectal, vaginal, dry powder inhaler or other inhalation or insufflation means. Alternatively, the composition may be presented in a form suitable for once-weekly or once-monthly administration; for example, an insoluble salt of the active compound, such as the decanoate salt, may be adapted to provide a depot preparation for intramuscular injection.

For preparing solid pharmaceutical compositions such as tablets, the principal active ingredient is mixed with a pharmaceutical carrier, e.g. conventional tableting ingredients such as diluents, binders, adhesives, disintegrants, lubricants, antiadherents and gildants. Suitable diluents include, but are not limited to, starch (i.e. corn, wheat, or potato starch, which may be hydrolized), lactose (granulated, spray dried or anhydrous), sucrose, sucrose-based diluents (confectioner's sugar; sucrose plus about 7 to 10 weight percent invert sugar; sucrose plus about 3 weight percent modified dextrins; sucrose plus invert sugar, about 4 weight percent invert sugar, about 0.1 to 0.2 weight percent cornstarch and magnesium stearate), dextrose, inositol, mannitol, sorbitol, microcrystalline cellulose (i.e. AVICEL™ microcrystalline cellulose available from FMC Corp.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (i.e. alginic acid and salts thereof, magnesium aluminum silicate, hydroxyethylcellulose [i.e. TYLOSE™ available from Hoechst Celanese], polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like. Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like. Suitable gildants include, but are not limited to, talc, cornstarch, silica (i.e. CAB-O-SIL™ silica available from Cabot, SYLOID™ silica available from W.R. Grace/Davison, and AEROSIL™ silica available from Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Generally these carriers are mixed with the pharmaceutical active to form a solid preformulation composition containing a homogeneous mixture of the pharmaceutical active of the present invention, or a pharmaceutically acceptable salt thereof. Generally the preformulation will be formed by one of three common methods: (a) wet granulation, (b) dry granulation and (c) dry blending. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.1 mg to about 500 mg of the active ingredient of the present invention. The tablets or pills containing the novel compositions may also be formulated in multilayer tablets or pills to provide a sustained or provide dual-release products. For example, a dual release tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric materials such as shellac, cellulose acetate (i.e. cellulose acetate phthalate, cellulose acetate trimetllitate), polyvinyl acetate phthalate, hydroxypropyl methylcellulose phthalate, hydroxypropyl methylcellulose acetate succinate, methacrylate and ethylacrylate copolymers, methacrylate and methyl methacrylate copolymers and the like. Sustained release tablets may also be made by film coating or wet granulation using slightly soluble or insoluble substances in solution (which for a wet granulation acts as the binding agents) or low melting solids a molten form (which in a wet granulation may incorporate the active ingredient). These materials include natural and synthetic polymers waxes, hydrogenated oils, fatty acids and alcohols (i.e. beeswax, carnauba wax, cetyl alcohol, cetylstearyl alcohol, and the like), esters of fatty acids metallic soaps, and other acceptable materials that can be used to granulate, coat, entrap or otherwise limit the solubility of an active ingredient to achieve a prolonged or sustained release product.

The liquid forms in which the novel compositions of the present invention may be incorporated for administration orally or by injection include, but are not limited to aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable suspending agents for aqueous suspensions, include synthetic and natural gums such as, acacia, agar, alginate (i.e. propylene alginate, sodium alginate and the like), guar, karaya, locust bean, pectin, tragacanth, and xanthan gum, cellulosics such as sodium carboxymethylcellulose, methylcellulose, hydroxymethylcellulose, hydroxyethylcellulose, hydroxypropyl cellulose and hydroxypropyl methylcellulose, and combinations thereof, synthetic polymers such as polyvinyl pyrrolidone, carbomer (i.e. carboxypolymethylene), and polyethylene glycol; clays such as bentonite, hectorite, attapulgite or sepiolite; and other pharmaceutically acceptable suspending agents such as lecithin, gelatin or the like. Suitable surfactants include but are not limited to sodium docusate, sodium lauryl sulfate, polysorbate, octoxynol-9, nonoxynol-10, polysorbate 20, polysorbate 40, polysorbate 60, polysorbate 80, polyoxamer 188, polyoxamer 235 and combinations thereof. Suitable deflocculating or dispersing agent include pharmaceutical grade lecithins. Suitable flocculating agent include but are not limited to simple neutral electrolytes (i.e. sodium chloride, potassium, chloride, and the like), highly charged insoluble polymers and polyelectrolyte species, water soluble divalent or trivalent ions (i.e. calcium salts, alums or sulfates, citrates and phosphates (which can be used jointly in formulations as pH buffers and flocculating agents). Suitable preservatives include but are not limited to parabens (i.e. methyl, ethyl, n-propyl and n-butyl), sorbic acid, thimerosal, quaternary ammonium salts, benzyl alcohol, benzoic acid, chlorhexidine gluconate, phenylethanol and the like. There are many liquid vehicles that may be used in liquid pharmaceutical dosage forms, however, the liquid vehicle that is used in a particular dosage form must be compatible with the suspending agent(s). For example, nonpolar liquid vehicles such as fatty esters and oils liquid vehicles are best used with suspending agents such as low HLB (Hydrophile-Lipophile Balance) surfactants, stearalkonium hectorite, water insoluble resins, water insoluble film forming polymers and the like. Conversely, polar liquids such as water, alcohols, polyols and glycols are best used with suspending agents such as higher HLB surfactants, clays silicates, gums, water soluble cellulosics, water soluble polymers and the like. For parenteral administration, sterile suspensions and solutions are desired. Liquid forms useful for parenteral administration include sterile solutions, emulsions and suspensions. Isotonic preparations which generally contain suitable preservatives are employed when intravenous administration is desired.

Furthermore, compounds of the present invention can be administered in an intranasal dosage form via topical use of suitable intranasal vehicles or via transdermal skin patches, the composition of which are well known to those of ordinary skill in that art. To be administered in the form of a transdermal delivery system, the administration of a therapeutic dose will, of course, be continuous rather than intermittent throughout the dosage regimen.

Compounds of the present invention can also be administered in the form of liposome delivery systems, such as small unilamellar vesicles, large unilamellar vesicles, multilamellar vesicles and the like. Liposomes can be formed from a variety of phospholipids, such as cholesterol, stearylamine, phosphatidylcholines and the like.

Compounds of the present invention may also be delivered by the use of monoclonal antibodies as individual carriers to which the compound molecules are coupled. The compounds of the present invention may also be coupled with soluble polymers as targetable drug carriers. Such polymers can include, but are not limited to polyvinylpyrrolidone, pyran copolymer, polyhydroxypropylmethacrylamidephenol, polyhydroxyethylaspartamidephenol, or polyethyl eneoxidepolylysine substituted with palmitoyl residue. Furthermore, the compounds of the present invention may be coupled to a class of biodegradable polymers useful in achieving controlled release of a drug, for example, to homopolymers and copolymers (which means polymers containing two or more chemically distinguishable repeating units) of lactide (which includes lactic acid d-, l- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, γ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, polyorthoesters, polyacetals, polydihydropyrans, polycyanoacrylates and cross-linked or amphipathic block copolymers of hydrogels and blends thereof.

Compounds of this invention may be administered in any of the foregoing compositions and dosage regimens or by means of those compositions and dosage regimens established in the art whenever treatment of integrin mediated disorders is required for a subject in need thereof.

The daily dose of a pharmaceutical composition of the present invention may be varied over a wide range from about 0.7 mg to about 21,000 mg per adult human per day; preferably, the dose will be in the range of from about 0.7 mg to about 7000 mg per adult human per day; most preferably the dose will be in the range of from about 0.7 mg to about 2100 mg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated. An effective amount of the drug is ordinarily supplied at a dosage level of from about 0.01 mg/kg to about 300 mg/kg of body weight per day. Preferably, the range is from about 0.01 mg/kg to about 100 mg/kg of body weight per day; and, most preferably, from about 0.01 mg/kg to about 30 mg/kg of body weight per day. Advantageously, a compound of the present invention may be administered in a single daily dose or the total daily dosage may be administered in divided doses of two, three or four times daily.

Optimal dosages to be administered may be readily determined by those skilled in the art, and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level.

Abbreviations used in the instant specification, particularly the Schemes and Examples, are as follows:
BSA Bovine Serum Albumen
DBC 2,6-Dichlorobenzoylchloride
DCM Dichloromethane
DIEA Diisopropylethylamine
DMF N,N-Dimethylformamide
EDAC N-ethyl-N′-dimethylaminopropylcarbodiimide hydrochloride
Et₂O Diethyl ether
EtOAc Ethyl acetate
EtOH Ethanol
h hour
HATU O-(7-azabenzotriazol-1-yl)-1,1,3,3-tetramethyluronium hexafluorophosphate
HPLC High Performance Liquid Chromatography
Me Methyl
MeOH Methanol
min Minutes
PBS Phosphate Buffer Solution
Ph Phenyl
rt Room temperature
SDS Sodium Dodecasulfate
THF Tetrahydrofuran
Thi Thienyl
TMS Tetramethylsilane
TFA Trifluoroacetic acid
Tol Toluene

### General Synthetic Methods

Representative compounds of the present invention can be synthesized in accordance with the general synthetic methods described below and are illustrated more particularly in the scheme that follows. Since the scheme is an illustration, the invention should not be construed as being limited by the chemical reactions and conditions expressed. The preparation of the various starting materials used in the schemes is well within the skill of persons versed in the art.

Scheme A describes a general synthetic method whereby intermediate and target compounds of the present invention may be prepared. Additional representative compounds and stereoisomers, racemic mixtures, diastereomers and enantiomers thereof can be synthesized using the intermediates prepared in accordance with the Schemes A and other materials, compounds and reagents known to those skilled in the art. All such compounds, stereoisomers, racemic mixtures, diastereomers and enantiomers thereof are intended to be encompassed within the scope of the present invention. Since the scheme is an illustration, the invention should not be construed as being limited by the chemical reactions and conditions expressed. The preparation of the various starting materials used in the scheme is well within the skill of persons versed in the art.

In the following general method for preparing compounds of the invention, amino ester Compound A1 is protected with a conventional amino protecting group to give Compound A2, which is saponified under basic conditions to yield carboxylic acid Compound A3. Compound A3 is condensed with Compound A4 in the presence of an appropriate coupling agent, base, and solvent. An appropriate coupling agent may include, and is not limited to, EDAC hydrochloride, DIC, EDC, DCC or HATU; an appropriate base may include but is not limited to, DIEA; and an appropriate solvent may include, but is not limited to, DCM or DMF. For compounds of the present invention, (S)-4-Nitrophenylalanine methyl ester is acylated with Compound A3 in the presence of EDC, HOBt, and DIEA in DCM.

The nitro group of Compound A5 is reduced with zinc powder to afford the correspondng amine, Compound A6. The amine of Compound A6 is acylated with a variety of acid chlorides to provide a variety of amides, represented by Compound A7. For example RA may be selected from the group consisting of —R₁₀, —R₁₂, —N(R₁₁,R₁₀), —N(R₁₁,R₁₂), and —N(R₁₂,R₁₇). Sulfamides could be made by analogues procedures. Compound A7 is deprotected under acidic conditions to provide the resultant amino Compound A8 which is then acylated by several methods: Compound A8 may be condensed with carboxylic acids in the presence of an appropriate coupling agent, base, and solvent. For example, R_{B} may be selected from the group consisting of R₃ which is aryl optionally substituted with one to three substituents independently selected from the group consisting of halogen, methyl, methoxy, carboxyl, aryl, amino, N-methylamino, N,N-dimethylamino, —CF₃ and —OCF₃; wherein the aryl substituent is optionally substituted with one to three substituents sleclected from the group consisting of halogen, methyl, methoxy, carboxyl, amino, N-methylamino, N,N-dimethylamino, —CF₃ and —OCF₃; or R₄ which is selected from the group consisting of C₁₋₆alkyl, C₂₋₆alkenyl, C₂₋₆alkynyl, and (halo)₁₋₃(C₁₋₆)alkyl; wherein C₁₋₆alkyl, C₂₋₆alkenyl and C₂₋₆alkynyl are optionally substituted on a terminal carbon with one substituent independently selected from R₁₄ Compounds of the present invention were made in the presence of HOBt, EDC, NMM in DCM; similarly, Compound A8 may be condensed with an appropriate acid chloride to provide Compound A9. The ester of Compound A9 is saponified under basic conditions to yield Compound A10. 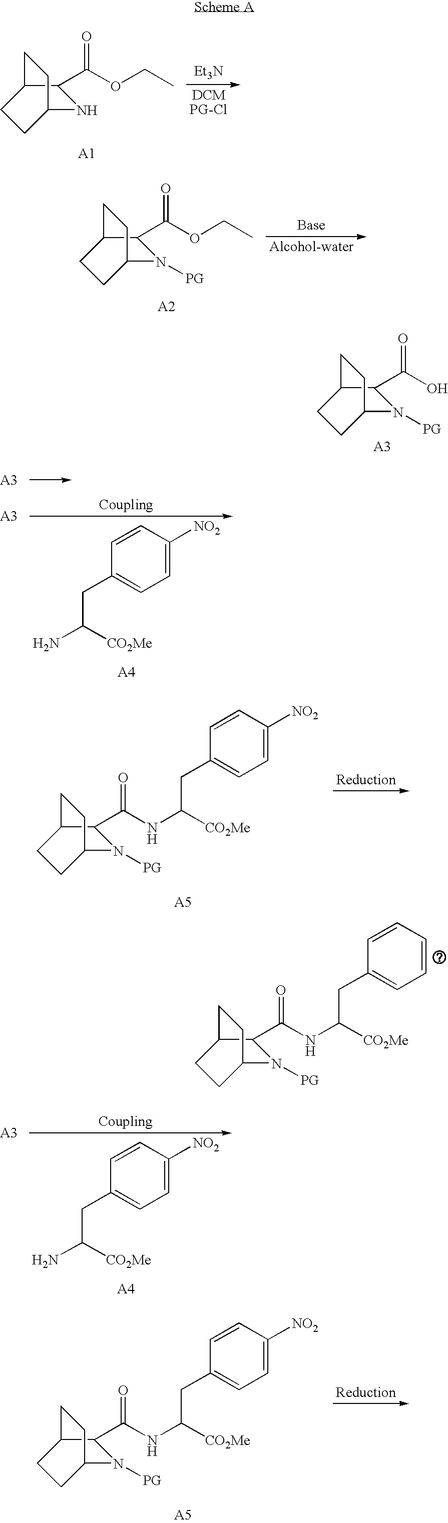 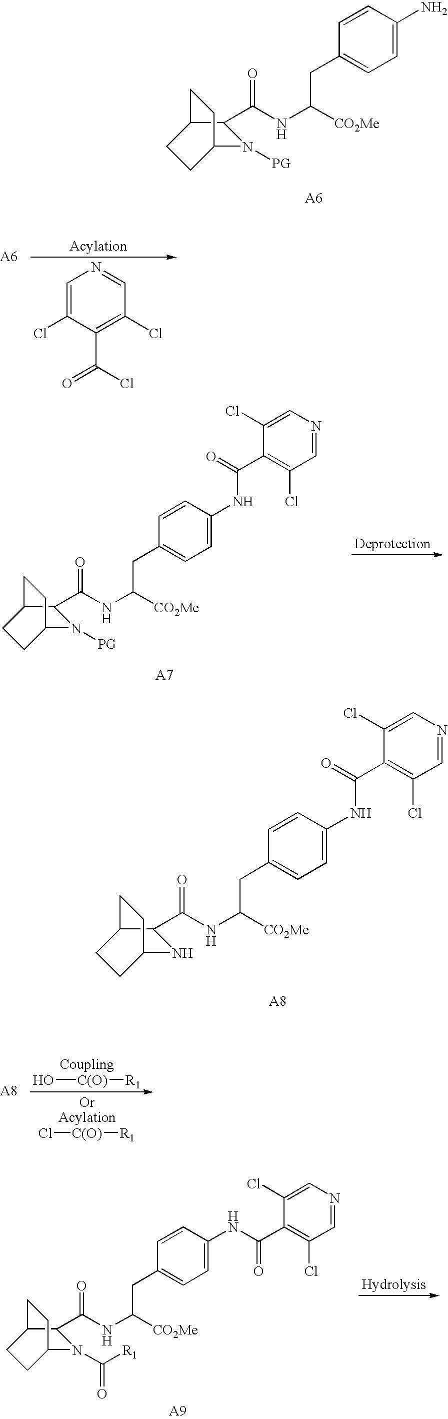 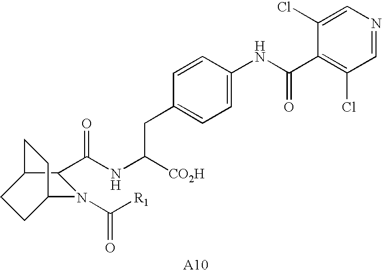
Preparation of ureas and alkyl-derivatives is shown on the Schemes B and C. 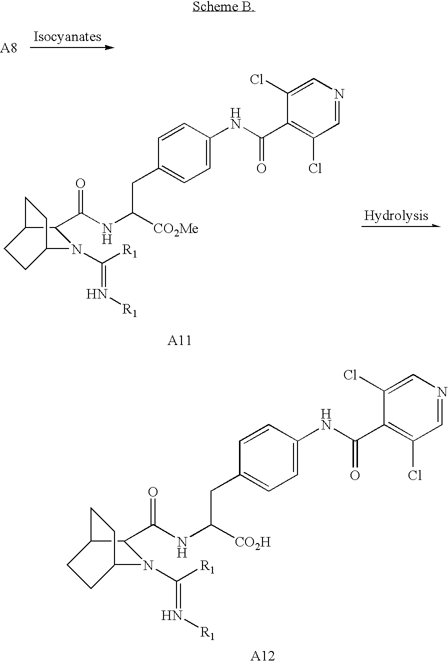
Treatment of A8 by isocyanate followed by hydrolysis resulted target compound A12. 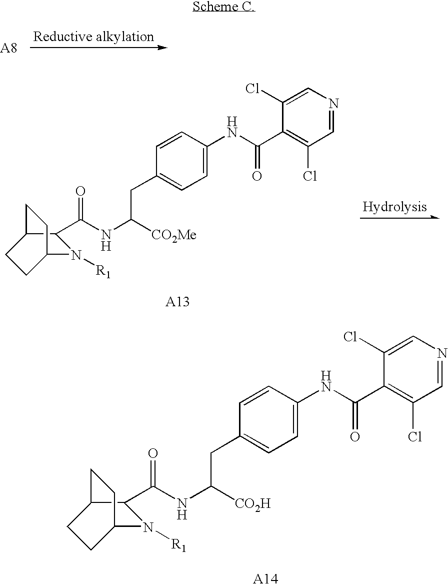
Reductive alkylation of A8 followed by hydrolysis provided target compound A14.

### Specific Synthetic Methods

Specific compounds which are representative of this invention were prepared as per the following examples and reaction sequences; the examples and the diagrams depicting the reaction sequences are offered by way of illustration, to aid in the understanding of the invention and should not be construed to limit in any way the invention set forth in the claims which follow thereafter. The instant compounds may also be used as intermediates in subsequent examples to produce additional compounds of the present invention. No attempt has been made to optimize the yields obtained in any of the reactions. One skilled in the art would know how to increase such yields through routine variations in reaction times, temperatures, solvents and/or reagents.

Reagents were purchased from commercial sources. Microanalyses were performed at Robertson Microlit Laboratories, Inc., Madison, N.J. and are expressed in percentage by weight of each element per total molecular weight. Nuclear magnetic resonance (NMR) spectra for hydrogen atoms were measured in the indicated solvent with (TMS) as the internal standard on a Bruker AM-360 (360 MHz) spectrometer. The values are expressed in parts per million down field from TMS. The mass spectra (MS) were determined on a Micromass Platform LC spectrometer using electrospray techniques as either (ESI) m/z (M+H⁺) or (ESI) m/z (M−H⁻). Stereoisomeric compounds may be characterized as racemic mixtures or as separate diastereomers and enantiomers thereof using X-ray crystallography and other methods known to one skilled in the art. Unless otherwise noted, the materials used in the examples were obtained from readily available commercial suppliers or synthesized by standard methods known to one skilled in the art of chemical synthesis. The substituent groups, which vary between examples, are hydrogen unless otherwise noted.

### EXAMPLE 1

### 2-Aza-bicyclo[2.2.2]octane-2-benzyloxycarbonyl-3-carboxylic acid ethyl ester

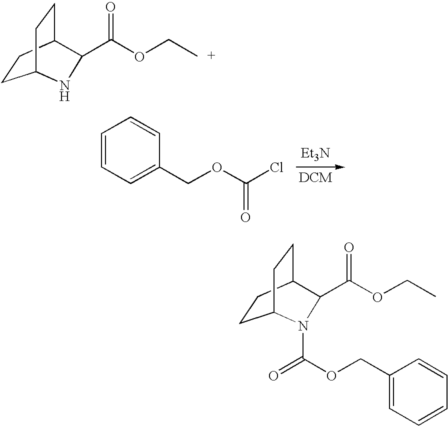

Amino ester (5.90 g, 0.0322 mol) was dissolved in 100 ml of dry DCM containing 9.43 mL (0.067 mol) of TEA and solution was cooled in the ice bath. Benzyl chloroformate (5.77 g, 4.83 mL, 0.0338 mol) was added dropwise (approximately 45 min). The reaction was stirred 2 hr at 0° C., then warmed up to RT and stirred overnight. The reaction mixture was washed with 0.1 N HCl, 5% NaHCO₃, water, dried over MgSO₄ and concentrated, resulting viscous oil. Product was analyzed by TLC (hexane:EtOAc 1:1, R_{f} 0.75). The crude material was purified by column chromatography (silica, hexane:EtOAc 7:1) resulting 7.73 g (76%) of pure product (viscous oil).

¹H NMR (300 MHz, CDCl₃): δ 7.37-7.26 (5H, m), 5.20-5.10 (2H, m), 4.71-4.69 (1H, m), 4.26-4.00 (3H, m), 2.24-2.22 (1H, m), 2.16-2.15 and 2.13-2.04 (1H, m), 2.00-1.40 (m, 9H), 1.25 and 1.15 (3H, J=7.3 Hz).

### EXAMPLE 2

### 2-Aza-bicyclo[2.2.2]octane-2-benzyloxycarbonyl-3-carboxylic acid

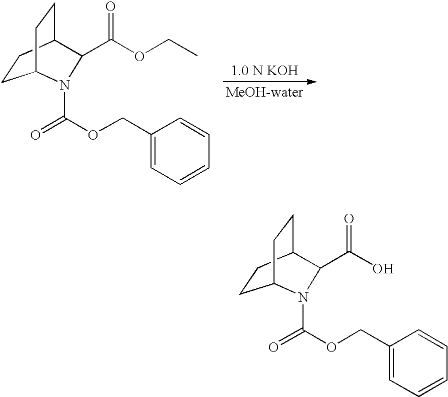

Ester (7.73 g, 24.36 mmol) was dissolved in 200 ml MeOH and 5 eq. of 1.0 N KOH aq. (122 mL) was added as one portion. Reaction was warmed up to 70° C. and stirred for 10 hr, then MeOH was evaporated. The residue was dissolved in 100 mL water, acidified by 1N HCl to pH 2 and extracted by EtOAc (3×100 mL). Organic fractions were combined, dried over MgSO₄, filtered and evaporated, providing white solid material (6.34 g, 90%).

¹H NMR (300 MHz, CDCl₃): δ 7.34-7.26 (5H, m), 5.29-5.10 (2H, m), 4.70-4.13 (2H, m), 2.29-2.23 (1H, m), 2.09-2.00 (1H, m), 2.00-1.40 (8H, m).

MS(ES⁻) 288.

### EXAMPLE 3

### 3-[1-Methoxycarbonyl-2-(4-nitro-phenyl)-ethylcarbamoyl]-2-aza-bicyclo [2.2.2]octane-2-carboxylic acid benzyl ester

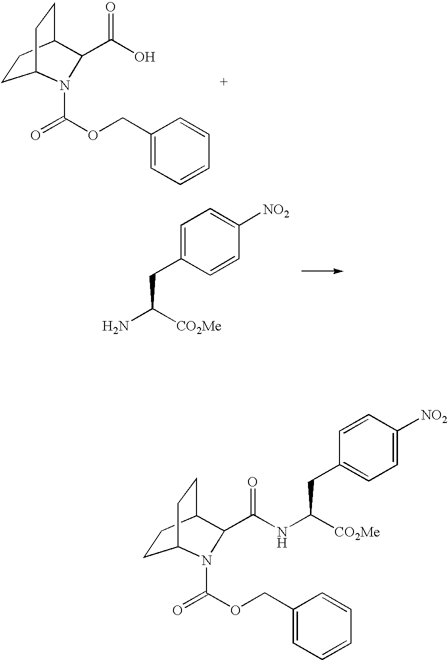

The acid (2.89 g, 10 mmol) was dissolved in 50 mL of dry DCM containing 2.11 g (11 mmol, 1.1 eq.) of EDC, 1.42 g (11 mmol, 1.1 eq.) of HOBt and 2.23 g (2.42 mL, 2.2 eq.) of DIEA. The amino ester (2.60 g, 1 eq.) was added as one portion, and reaction was stirred under nitrogen atmosphere for 3 hr at RT. The reaction mixture was washed with water (100 mL), 10% citric acid solution, 5% NaHCO₃ aq., dried over MgSO₄, filtered and evaporated. The residue (yellow foam, 4.58 g) was purified by column chromatography (silica, eluent hexane:EtOAc 1:1; R_{f} 0.59) providing 3.78 g (76%) of pure product.

¹H NMR (300 MHz, CDCl₃): δ 8.13 and 8.11 (2H, s), 7.40-7.00 (7H, m), 6.80-6.60 (1H, broad s), 5.17-5.12 (2H, broad s), 4.95-4.89 (1H, m), 4.14-4.00 (1H, m), 3.72 and 3.59 (3H, s), 3.35-3.05 (2H, m), 2.10-2.02 (1H, m), 2.00-1.20 (8H, m)

MS(ES⁺) 496

### EXAMPLE 4

### 3-[2-(4-Amino-phenyl)-1-methoxycarbonyl-ethylcarbamoyl]-2-aza-bicyclo[2.2.2]octane-2-carboxylic acid benzyl ester

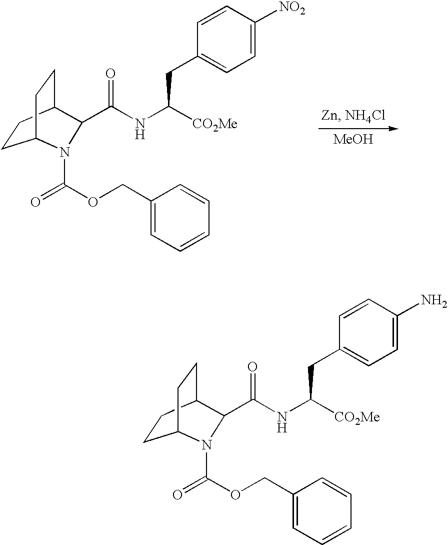

The solution of nitro compound (3.54 g, 7.14 mmol) in MeOH (70 mL) was placed in the round-bottom flask equipped with mechanical stirrer and reflux condenser, containing Zn powder (4.67 g, 71.4 mmol, 10 eq.) and NH₄Cl (1.91 g, 35.7 mmol, 5 eq.). The mixture was stirred for 3 hr at 65° then cooled to room temperature and filtered through celite. The clear solution was concentrated to approximately 20 mL, diluted by 200 mL of 10% NaHCO₃ aq. and extracted by EtOAc (4×20 mL). Organic fractions were combined, dried over MgSO₄ filtered and evaporated. The residue was subjected to column chromatography (silica, EtOAc:hexane 1:1, R_{f} 0.11) resulting 3.19 g of yellow solid (96%).

¹H NMR (300 MHz, CDCl₃): δ 7.35-7.10 (7H, m), 6.90-6.75 (2H, m), 6.57 (2H, d, J=8.3 Hz), 6.55 (1H, m), 5.15-5.05 (2H, m), 4.85-4.81 (1H, m), 4.15-4.08 (2H, m), 3.69-3.59 (3H, m), 3.00-2.93 (2H, m), 2.1-2.00 (1H, m), 1.90-1.20 (8H, m).

MS(ES⁺) 466

### EXAMPLE 5

### 3-(2-{4-[(3,5-Dichloro-pyridine-4-carbonyl)-amino]-phenyl}-1-methoxycarbonyl-ethylcarbamoyl)-2-aza-bicyclo[2.2.2]octane-2-carboxylic acid benzyl ester

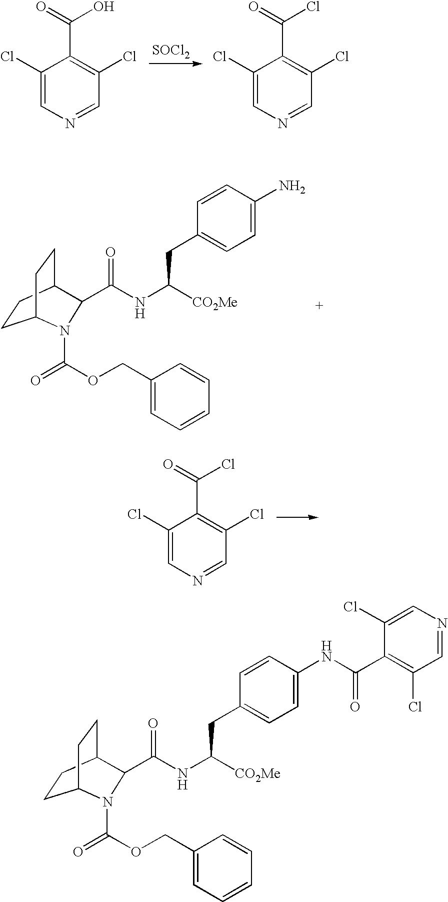 Preparation of the 3,5-dichloro-isonicotinoyl chloride.

The acid (1.50 g, 7.81 mmol) was placed in the round-bottom flask equipped with stirrer and reflux condenser. DCM (20 mL) containg 2 drops of DMF was added as one portion followed by thionyl chloride (1.40 g, 0.85 ml, 1.5 eq.). The reaction mixture was refluxed for 3 hr resulting clear solution. The solution was evaporated in vacuum providing yellow oil, which was used in the next step without purification.

Acylation Reaction

The aniline (2.88 g, 6.186 mmol) was dissolved in 50 ml of DCM, containing TEA (1.619 g, 2.23 mL, 16 mmol) and placed in the round-bottom flask equipped with mechanical stirrer and immersed in the ice bath. The solution of the acid chloride (step A) in 20 mL DCM was added dropwise (45 min), reaction was allowed to warm up to RT and stirred overnight. The solution was washed with NaHCO3 10%, 0.1 N HCl, water, dried over MgSO4 and evaporated. The residue was subjected to column chromatography (eluent EtOAc) 3.65 g (92%) of yellow solid material.

¹H NMR (300 MHz, CD₃CN): δ 8.90 (1H, s), 8.65 (2H, s), 7.65-7.50 (2H, m), 7.45-7.15 (9H, m), 6.97 (1H, d, J=8.0 Hz), 5.12-4.99 (2H, m), 4.75-4.60 (1H, m), 4.09-4.01 (2H, m), 3.69-3.62 (3H, m), 3.20-2.90 (1H, m),

MS(ES⁺) 639

### EXAMPLE 6

### 2-[(2-Aza-bicyclo[2.2.2]octane-3-carbonyl)-amino]-3-{4-[(3,5-dichloro-pyridine-4-carbonyl)-amino]-phenyl}-propionic acid methyl ester

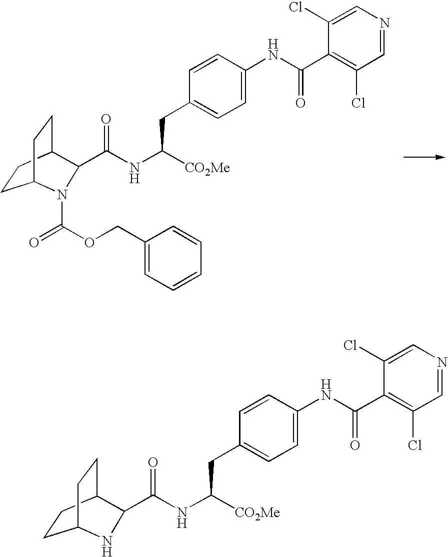

Benzyloxycarbonyl-derivative (3.65 g, 5.72 mmol) was added to 45 ml of 33% HBr in AcOH under vigorous stirring. The reaction was kept at RT for 3 hr (reaction became homogeneous after 45 min). The viscous liquid was evaporated in vacuum, the residue was dissolved in water (250 ml) and extracted with ether, the organic layer was discarded. Aqueous layer was basified to pH 7 with Na₂CO₃ and extracted with EtOAc (5×20 mL). The organic layers were combined, dried over Na₂SO₄ and evaporated, providing 2.85 g of slightly yellow solid. The residue was subjected to column chromatography (CHCl₃:MeOH 9:1) providing 2.5 g (87%) of white solid.

¹H NMR (300 MHz, DMSO-d₆): δ 8.80 (2H, s), 8.48 (1H, d, J=8.3 Hz), 7.57 (2H, d, J=8.5 Hz), 7.21 (2H, d, J=8.5 Hz), 4.69-4.61 (1H, m), 3.66 (3H, s), 3.13-3.00 (2H, m), 2.76 (2H, m), 2.50 (1H, broad s), 1.84 (1H, broad s), 1.62-1.08 (9H, m).

MS(ES⁺) 505

Preparation of the Target Amide

1 Acylation Reaction

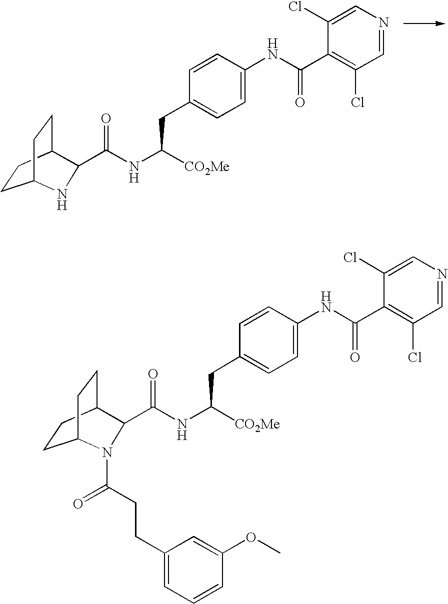

The amine (50.5 mg, 0.10 mmol, 1 eq.), 3-methoxyphenyl propanoic acid (17.4 mg, 0.105 mmol, 1.05 eq.), EDC hydrochloride (21.1 mg, 0.11 mmol, 1.1 eq.), HOBt (14.2 mg, 0.105 mmol, 1.05 eq.) were suspended in 1 mL DCM at RT and N-Me morpholine (13.2 mL, 1.20 eq.) was added as one portion. The reaction was kept at RT for 4 hr and loaded in to the silica column. Flash chromatography (eluent EtOAc) provided 39 mg (59%) of white solid material.

MS(ES⁺) 667
2 Hydrolysis Reaction

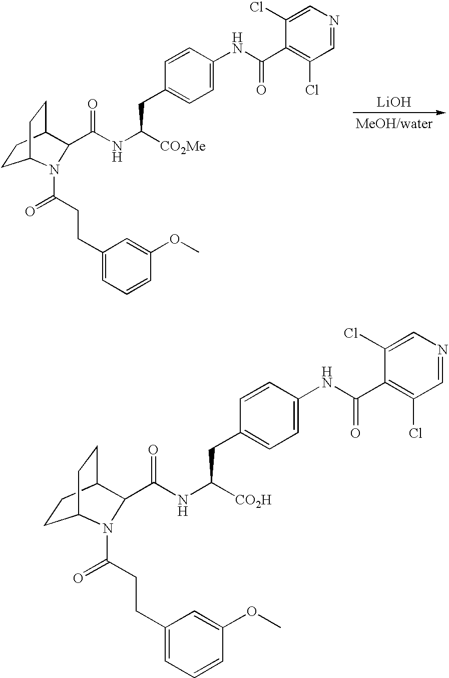

The Me ester (39 mg, 0.06 mmol) was dissolved in 2 ml MeOH:water 1:1 mixture and LiOH hydroxide (4 mg, 1.6 eq.) was added as portion. Reaction was homogenized in the ultrasonic bath and kept overnight at RT. The reaction mixture was diluted by 20 mL of water, extracted by 10 mL of ether and organic layer was discarded. The aqueous layer was acidified by 1 N HCl to pH 2 and extracted by EtOAc (2×10 mL). Organic layers were combined, dried over MgSO4, filtered and evaporated providing white residue which was purified by HPLC. After lyophilization 25 mg of TFA salt were obtained.

¹H NMR (300 MHz, DMSO-d₆): δ 10.87 and 10.81 (1H, s), 8.79 and 8.77 (2H, s), 8.46 and 7.95 (1H, d, J=8.4 Hz), 7.56 and 7.53 (2H, d, J=5.8 Hz), 7.29-7.26 (2H, m), 7.15 (1H, q, J=9.1 Hz), 6.89-6.61 (3H, m), 4.71-4.66 and 4.45-4.35 (1H, m), 4.22 and 4.21 (1H, broad s), 4.05 and 3.97 (1H, broad s), 3.73 and 3.71 (3H, s), 3.72-3.60 (1H, m), 3.30-3.15 (2H, m), 3.04-2.88 (2H, m), 2.10 and 2.04 (2H, m), 1.85-1.10 (9H, m).

MS(ES⁺) 653

### EXAMPLE 7

### Procedure for preparation of 3-{4-[(3,5-dichloro-pyridine-4-carbonyl)-amino]-phenyl}-2-[(2-p-tolylcarbamoyl-2-aza-bicyclo[2.2.2]octane-3-carbonyl)-amino]-propionic acid

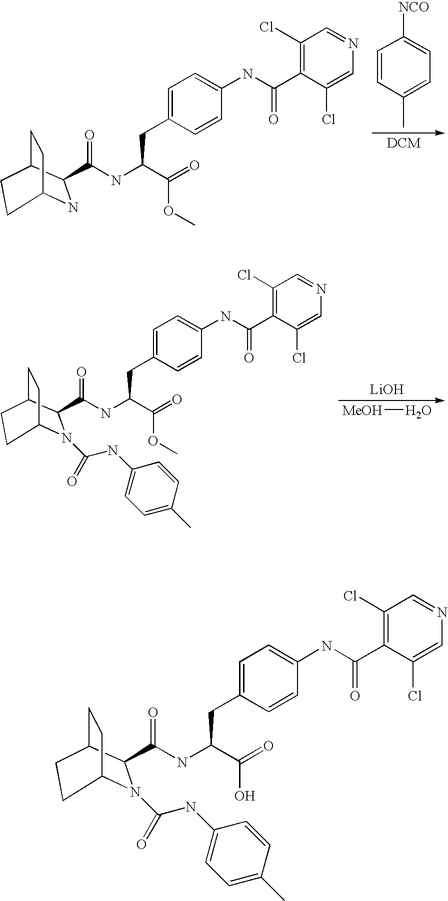

4-Tolyl isocyanate (0.012 mL, 0.095 mmol) was added to bicyclic scaffold (35 mg, 0.070 mmol) in dicholoromethane (1.5 mL). The mixture was stired at r.t. for 1 h, and then concentrated. The crude ester was dissolved in MeOH (1 mL) and treated with 1N LiOH (0.20 mL, 0.20 mmol) at r.t. for 4 h. The mixture was filtered. The filtrate was concentrated and treated with H₂O—CH₂Cl₂. The aqueous layer was washed with CH₂Cl₂, and acidified with 2N HCl. The white solid was collected, washed with H₂O three times, and dried (28 mg). ¹H NMR (CD₃OD) δ: 8.62 (s, 2H), 7.57 (d, 2H), 7.30 (d, 2H), 7.21 (d, 2H), 7.03 (d, 2H), 4.22 (d, 1H), 4.02 (d, 1H), 3.11 (m, 1H), 2.25 (s, 3H), 2.20 (m, 1H), 1.98-1.20 (br, 9H). LC/MS: M+1=624.

### EXAMPLE 8

### Procedure for the preparation of 3-{4-[(3,5-dichloro-pyridine-4-carbonyl)-amino]-phenyl}-2-{[2-(3,3-dimethyl-butyl)-2-aza-bicyclo[2.2.2]octane-3-carbonyl]-amino}-propionic acid

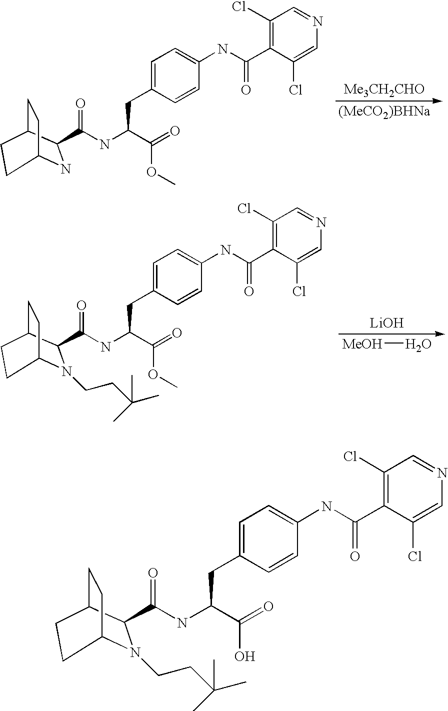

A 10-mL vial (SmithProcess) containing a magnetic stir bar was charged with bicyclic scaffold (0.30 g, 0.60 mmol), Me₃CH₂CHO (0.11 mL, 0.88 mmol), acetic acid (40 μL), sodium triacetoxyborohydride (0.18 g 0.85 mmol) in ethylene dichloride (2.0 mL). The vial was sealed and the mixture was heated under microwave (SmithSynthesizer) at 120° C. for 5 min. The reaction mixture was concentrated and treated with 1N LiOH (1.5 mL) in MeOH (6.0 mL) at room temperature for 6 h. The mixture was acidified with TFA and purification by HPLC giving desired compound as a white solid (0.31 g). ¹H NMR (CD₃OD) δ: 8.64 (s, 2H), 7.57 (d, 2H), 7.27 (d, 2H), 4.59 (m, 1H), 3.70 (br, 1H), 3.3 (br, 2H), 3.01 (br, 2H), 2.37-1.17 (br, 12H), 0.90 (s, 9H). LC/MS: M+1=575.

### EXAMPLE 9

Alternative method of intermediate preparation: convergent approach

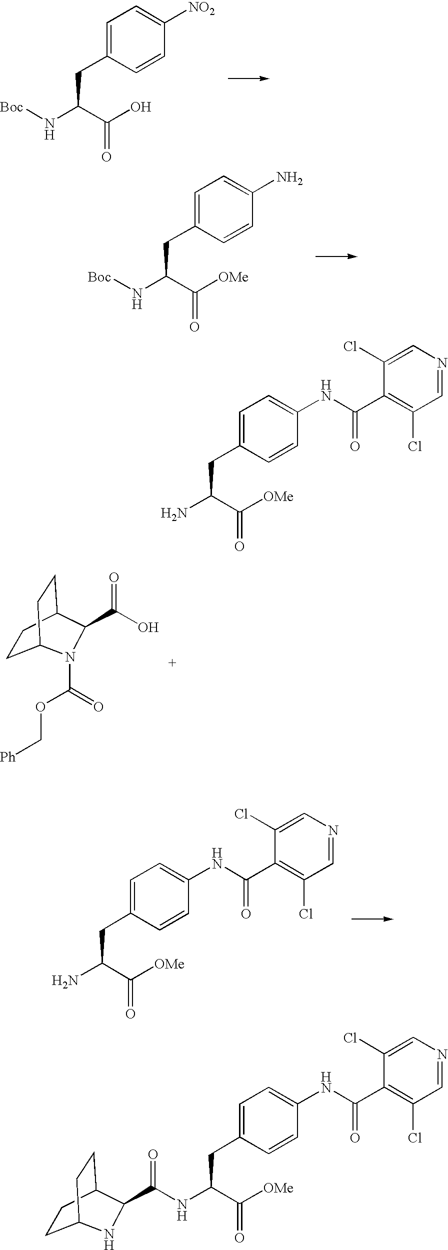

N-Boc-4-nitro-L-phenylalanine (5 g) was dissolved in 100 ml MeOH-chloroform 1:1 mixture, the solution was cooled in the ice bath. Trimethylsilyl diazomethane (1 M solution in hexane) was added dropwise until the solution remained yellow. The reaction mixture was evaporated in vacuum, the residue was dissolved in 50 ml MeOH-ethyl acetate 1:1 mixture and was hydrogenated at 30 psi overnight over Pd/C 10% (100 mg). After filtration the solvent was evaporated, providing 5.1 g of amino compound as white solid.

¹H NMR (CDCl₃) δ 6.89 (d, J=8.2 Hz, 2H), 6.60 (d, J=8.3 Hz, 2H), 5.05-4.95 (m, 1H), 4.59-4.50 (m, 1H), 3.01-2.95 (m, 2H), 1.41 (s, 9H); MS(ES⁺) 295.

Anal. calcd. for C₁₅H₂₂N₂O₄: C, 61.21; H, 7.53; N, 9.52. Found: C, 61.24; H, 7.80; N, 9.46.

The Me ester (5.0 g, 0.017 mol) was dissolved in 50 ml CH₂Cl₂ containing 3 ml of Et₃N followed by 5.31 g (0.025 mol) of 3,5-dichloroisonocotinoyl chloride. The reaction mixture was kept overnight at room temperature, washed 0.1 N HCl, 10% NaHCO₃, dried over MgSO₄, filtered and evaporated. The product was purified by crystallization from hexane/ethyl acetate, providing. 6.22 g (78% yield) of N-BOC-4-dichloroisonicotinamido phenylalanine methyl ester as white solid, mp 124-126° C.

¹H NMR (DMSO-d₆) δ 8.79 (s, 2H), 7.56 (d, J=8.4 Hz, 2H), 7.30 (d, J=8.1 Hz, 1H), 7.24 (d, J=8.4 Hz, 2H), 4.17-4.13 (m, 1H), 2.97 (dd, J=13.7 and 5.0, 1H), 2.83 (dd, J=13.6 and 9.9, 1H); 1.33 (s, 9H); MS (ESI+) 469.

Anal. calcd. for C₂₁H₂₃Cl₂N₃O₅.0.8Et₂O: C, 55.09; H, 5.92; N, 7.96. Found: C, 54.94; H, 5.86; N, 8.00.

N-BOC-4-dichloroisonicotinamido phenylalanine methyl ester (4.68 g, 0.01 Mol) was dissolved in 30 ml of CH₂Cl₂ followed by 1 ml of TFA. Reaction was kept overnight at room temperature, evaporated in vacuum, and the viscous residue was recrystallized from CH₂Cl₂/ether, providing TFA salt of the free amine as white solid (5.1 g, 80% yield); mp 257-259° C.

¹H NMR (DMSO-d₆): δ 8.80 (s, 2H), 8.44 (broad s, 3H), 7.63 (d, J=8.4 Hz, 2H), 7.63 (d, J=8.5 Hz, 2H), 7.24 (d, J=8.5 Hz, 2H), 4.33 (t, J=6.4 Hz, 1H), 3.09 (d, J=6.4, 2H), MS (ESI+) 369.

Anal. calcd. for C₁₆H₁₅Cl₂N₃O₃CF₃CO₂H: C, 44.83; H, 3.34; N, 8.71. Found: C, 44.49; H, 3.23; N, 8.61.

### EXAMPLE 10

Several examples of the characterization of the final compounds

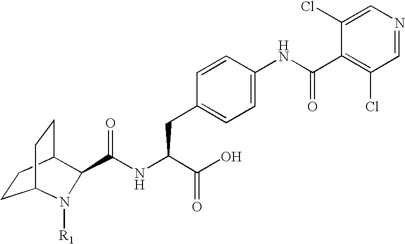

The following compounds were made following the procedures and schemes provided in the previous Examples.

26 (R¹=t-BuCO): ¹H NMR (dmso-d₆): [symbol] 10.80 (s, 1H); 8.73 (s, 2H), 7.71 (d, J=6.97 d, 1H), 7.49 (d, J=8.5 Hz, 2H), 7.21 (d, J=8.5 Hz, 2H), 4.23-4.28 (m, 1H), 4.09-4.05 (m, 2H), 3.00-2.80 (m, 2H), 2.03 (s, 1H), 1.90-1.73 (m, 1H), 1.65-1.40 (m, 6H), 1.35-1.15 (m, 1H), 1.08 (s, 9H). MS m/z 576 (MH+). Anal. calcd. (C₂₈H₃₂Cl₂N₄O₅.0.25 TFA) C, 56.67; H, 5.38; Cl, 11.74; N, 9.28. Found: C, 56.22; H, 5.01; Cl, 12.02; N, 9.02.

27(R¹=t-Bu-CH₂CO): (CD₃OD, rotamers): [symbol] 8.65 (s, 2H), 7.60-7.55 (m, 2H), 7.34-7.25 (m, 2H), 4.71 (m, 0.5H), 4.46 (m, 0.5H), 4.32 (m, 0.5H), 4.26 (m, 0.5H), 4.03 (m, 1H), 3.20-2.90 (m, 2H), 2.20-1.20 (m, 12H), 1.05 and 0.94 (s, 9H). MS m/z 589 (MH+). Anal. calcd. (C₂₉H₃₄Cl₂N₄O₅.1.2 TFA). C, 51.92; H, 4.88; F, 9.42; Cl, 9.76; N, 7.71. Found: C, 52.25; H, 4.80; F, 10.18; Cl, 10.14; N, 7.78.

38(R¹=i-Pr—(CH₂)₂CO): ¹H NMR (dmso-d₆, rotamers): [symbol] 10.74 and 10.72 (s, 1H); 8.71 (s, 2H), 7.42-7.35 (m, 2.5H), 7.14-7.06 (m, 2.5H), 4.24 (m, 0.5H), 4.08-4.00 (m, 1H), 3.98-3.95 (m, 0.5H), 3.87-3.85 (m, 0.5H), 3.79 (m, 0.5H), 3.00-2.90 (m, 2H), 2.32-2.22 (m, 0.5H), 2.16-2.05 (m, 0.5H), 2.05 and 2.00 (m, 1H), 1.96-1.89 (m, 0.5H), 1.85-1.75 (m, 0.5H), 1.65-1.15 (m, 11H), 0.80 (d, J=6.6 Hz, 3H), 0.72 (dd, J=6.4 and 3.7 Hz, 3H). MS m/z 589 (MH+). Anal. calcd. (C₂₉H₃₄Cl₂N₄O₅.2.5 TFA.0.5H₂O) C, 46.22; H, 4.28; Cl, 8.02; N, 6.34; F, 16.13. Found: C, 46.21; H, 4.34; Cl, 7.73; N, 6.46; F, 15.69.

39(R¹=Ph(CH₂)₂CO): ¹H NMR (dmso-d₆, rotamers): [symbol] 10.75 (s, 1H), 8.72 (s, 1H), 7.66 (m, 1H), 7.45-7.39 (m, 2H), 7.20-7.15 (m, 3H), 7.10-7.03 (m, 2H), 4.25 (m, 1H), 4.04 (m, 1H), 3.85-3.75 (m, 1H), 3.10-2.50 (m, 4H), 2.00-1.10 (m, 10H). MS m/z 623 (MH+). Anal. calcd. (C₃₂H₃₂Cl₂N₄O₅.1TFA) C, 55.37; H, 4.51; Cl, 9.61; N, 7.60. Found: C, 55.29; H, 4.16; Cl, 9.45; N, 7.42.

3(R¹=3-thienylCH₂CO): ¹H NMR (dmso-d6, rotamers): [symbol] 10.79 (s, 1H), 8.78 (m, 2H), 7.52-7.38 (m, 4H), 7.22-6.94 (m, 4H), 4.30-3.60 (m, 4H), 3.15-2.95 (m, 2H), 2.07 (m, 1H), 1.75-1.20 (m, 9H). MS m/z 615 (MH⁺). Anal. calcd. (C₂₉H₂₈Cl₂N₄O₅S₂H₂O, 1.5 TFA) C, 46.72; H, 4.10; N, 6.81; Cl 8.62; F, 10.39; S 3.90. Found: C, 46.24; H, 3.96; N, 6.61; Cl, 9.02; F, 10.07; S, 3.84; KF 4.06.

33(R¹=3,5-(MeO)₂PhCH₂CO): 1H NMR (CD₃OD, rotamers): [symbol] 8.64-8.63(m, 2H), 7.59-7.52 (m, 2H), 7.32-7.28 (m, 2H), 6.92-6.75 (m, 3H), 4.70-4.73 (m, 1H), 4.43-4.21 (m, 1H), 3.91 (s, 1H), 3.81-3.72 (m,6H), 3.61-3.48 (m, 1H), 3.39-3.31 (m, 1H), 3.16-2.91 (m, 2H), 2.17 (s, 1H), 1.93-1.45 (m, 8H). MS m/z 669 (MH⁺). Anal. calcd. (C₃₃H₃₄Cl₂N₄O₇.1H₂O, 1 TFA) C, 52.44; H, 4.65; N, 6.99; F, 7.11. Found: C, 52.20; H, 4.10; N, 6.59; F, 6.56.

3(R¹⁼²-pyridyl-CH₂CO): ¹H NMR (CD₃OD, rotamers): [symbol] 8.79-8.71 (m, 1H), 8.62 (m, 2H), 8.47-8.34 (m, 1H), 7.92-7.77 (m, 1.5H), 7.67 (m, 0.5H), 7.52-7.42 (m, 2H), 7.33-7.21 (m, 2H), 4.68-4.62 (m, 1H), 4.42-4.30 (m, 1H), 4.00-3.95 (m, 1H), 3.56-3.29 (m, 1H), 3.17-2.92 (m, 3H), 2.30-2.17 (m, 1H), 2.08-1.15 (m, 8H). MS m/z 610 (MH⁺). Anal. calcd. (C₃₀H₂₉Cl₂N₅O₅.1.5H₂O, 1.3 TFA) C, 49.83; H, 4.27; N, 8.91; F, 9.43. Found: C, 49.92; H, 3.78; N, 8.87; F, 9.68.

44(R¹=4-pyridyl-CH₂CO): ¹H NMR (CD₃OD, rotamers): δ 8.78-8.26 (m, 5H), 8.00-7.93 (m, 1H), 7.56-7.48 (m, 2H), 7.35-7.29 (m, 2H), 4.70-4.67 (m, 1H), 4.41-4.35 (m, 1H), 4.21-4.17 (m, 1H), 4.05-3.95 (m, 1H), 3.70-3.34 (m, 1H), 3.20-2.97 (m, 2H), 2.28-2.20 (m, 1H), 2.03-1.13 (m, 8H). MS m/z 610 (MH⁺). Anal. calcd. (C₃₀H₂₉Cl₂N₅O₅.1H₂O, 1 TFA) C, 50.41; H, 4.19; N, 9.02; F, 9.54. Found: C, 50.26; H, 3.69; N, 8.69; F, 9.20.

51(R¹=4-Cl—Ph(CH₂)₂CO): (dmso-d₆, rotamers): δ 10.85 and 10.83 (s, 1H), 8.76 and 8.75 (s, 2H), 7.50-7.43 (m, 2H), 7.30-7.18 (m, 7H), 4.25 (m, 0.5H), 4.20 (dd, J=12.7 and 7.2 Hz, 0.5H), 4.05 (m, 0.5H), 4.20 (dd, J=7.2 and 5.2 Hz, 0.5H), 3.90 (m, 0.5H), 3.84 (m, 0.5H), 3.06-3.00 (m, 1H), 2.95-2.55 (m, 5H), 2.25-2.10 (m, 1H), 1.65-1.15(m, 9H). MS m/z 657 (MH⁺). Anal. calcd. (C₃₂H₃₁Cl₃N₅O₅.3.5H₂O, 3.5 TFA) C, 41.82; H, 3.73; N, 5.00; F, 17.81; Cl, 9.50; Karl Fisher 5.63. Found: C, 42.26; H, 3.23; N, 4.46; F, 18.02; Cl, 9.07; Karl Fisher 5.35.

61(R¹=PhCH₂OCO): ¹H NMR (CD₃OD, rotamers): [symbol] 8.63 (s, 2H), 7.60-7.50 (m, 2H), 7.40-7.15 (m, 7H), 5.11 and 5.07 (m, 1H), 4.80-4.65 (m, 1H), 4.17 (m, 1H), 4.04 (m, 1H), 3.25-3.15 (m, 1H), 3.03-2.80 (m, 1H), 2.20-1.20 (m, 10H). MS m/z 626 (MH⁺). Anal. calcd. (C₃₂H₃₂Cl₂N₄O₅) C, 59.53; H, 4.83; Cl, 11.34; N, 8.96. Found: C, 59.26; H, 4.56; Cl, 11.59; N, 8.70.

48??(R¹=Ph(CH₂)₃): ¹H NMR (CD₃OD, rotamers): δ 8.65-8.61 (m, 2H), 7.65-7.55 (m, 2H), 7.31-7.08 (m, 7H), 3.81-3.72 (m, 1H), 3.58-3.31 (m, 3H), 3.16-2.78 (m, 2H), 2.75-2.48 (m, 2H), 2.23 (bs, 1H), 2.05-1.11 (m, 11H).). MS m/z 609 (MH⁺). Anal. calculated. (C₃₂H₃₄Cl₂N₅O₄.0.5H₂O, 1.6 TFA) C, 52.78; H, 4.61; N, 6.99; F, 11.38. Found: C, 52.72; H, 4.04; N, 6.97, F, 11.05.

17(R¹═Ph(CH₂)₂): 1H NMR (CD₃OD, rotamers): [symbol] 8.63 (m, 2H), 7.57 (M, 2H), 7.34-7.24 (m, 7H), 3.97 (m, 1H), 3.16-3.04 (m, 3H), 2.52-2.29 (m, 4H), 2.29 (bs, 1H), 2.20-1.50 (m, 9H). MS m/z 595 (MH⁺). Anal. calcd. (C₃₁H₃₂Cl₂N₄O₄.0.4H₂O, 1.4 TFA) C, 53.25; H, 4.52; N, 7.35; F, 10.47. Found: C, 52.92; H, 3.91; N, 7.48; F, 10.28.

18(R¹═PhCH₂): ¹H NMR (CD₃OD, rotamers): [symbol] 8.67 (s, 2H), 7.57 (m, 2H), 7.42 (m, 2H), 7.39-7.23 (m, 3H), 7.02 (m, 2H), 4.43-4.32 (m, 2H), 4.30-4.20 (m, 1H), 3.80 (s, 1H), 3.57 (m, 1H), 3.03-2.92 (m, 1H), 2.77-2.67 (m, 1H), 2.42-2.21 (m, H), 2.18-2.02 (m, 1H), 1.98-1.72 (m, 4H), 1.63-1.52 (m, 2H). Anal. calcd. (C₃₀H₃₀Cl₂N₄O₄.1.0H₂O, 1.6 TFA) C, 51.00; H, 4.33; N, 7.17; F, 11.66. Found: C, 50.93; H, 3.75: N, 7.12: F, 11.45.

### BIOLOGICAL EXPERIMENTAL EXAMPLES

### Example 11

As demonstrated by biological studies described hereinafter, and shown in Table III and Table IV, the compounds of the present invention are α4β1 and α4β7 integrin receptor antagonists useful in treating integrin mediated disorders including, but not limited to, inflammatory, autoimmune and cell-proliferative disorders.

Ramos Cell Adhesion Assay α₄β₁ Mediated Adhesion/VCAM-1) Immulon 9.6 well plates (Dynex) were coated with 100 μL recombinant hVCAM-1 at 4.0 μg/mL in 0.05 M NaCO₃ buffer pH 9.0 overnight at 4° C. (R&D Systems). Plates were washed 3 times in PBS with 1% BSA and blocked for 1 h @ room temperature in this buffer. PBS was removed and compounds to be tested (50 μL) were added at 2× concentration. Ramos cells, (50 μL at 2×10⁶/mL) labeled with 5 μM Calcein AM (Molecular Probes) for 1 h at 37° C., were added to each well and allowed to adhere for 1 h at room temperature. Plates were washed 3× in PBS+1% BSA and cells were lysed for 15 minutes in 100 μL of 1 M Tris pH 8.0 with 1% SDS. The plate was read at 485 nm excitation and 530 nm emission.

α₄β₇-K562 Cell Adhesion Assay (α₄β₇ Mediated Adhesion/VCAM-1) Immulon 96 well plates (Dynex) were coated with 100 μL recombinant hVCAM-1 at 4.0 μg/mL in 0.05 M NaCO₃ buffer pH 9.0 overnight at 4° C. (R&D System). Plates were washed 3 times in PBS with 1% BSA and blocked for 1 h @ room temperature in this buffer. PBS was removed and compounds to be tested (50 μL) were added at 2× concentration. A stable cell line of K562 cells expressing human α₄β₇, (50 μL at 2×10⁶/mL) labeled with 5 μM Calcein AM (Molecular Probes) for 1 h at 37° C., were added to each well and allowed to adhere for 1 h at room temperature. Plates were washed 3× in PBS+1% BSA and cells were lysed for 15 minutes in 100 μL of 1 M Tris pH 8.0 with 1% SDS. The plate was read at 485 nm excitation and 530 nm emission.

TABLE 5

| | α4β1 | α4β7 |
|---|---|---|
| Cpd | IC₅₀ (nM) | IC₅₀ (nM) |
| 1 | 156 | 93 |
| 2 | 164 | 316 |
| 3 | 43 | 41 |
| 4 | 25 | 32 |
| 5 | 20 | 62 |
| 6 | 234 | 387 |
| 7 | 63 | 44 |
| 8 | 188 | 1015 |
| 9 | 436 | 151 |
| 10 | 842 | 185 |
| 11 | 51 | 368 |
| 12 | 460 | 739 |
| 13 | >5000 | >5000 |
| 14 | >5000 | >5000 |
| 15 | 69 | 39 |
| 16 | 47 | 94 |
| 17 | 62 | 262 |
| 18 | 543 | 309 |
| 19 | 2560 | >5000 |
| 20 | 259 | 287 |
| 21 | 585 | 124 |
| 22 | 380 | 670 |
| 23 | 248 | 444 |
| 24 | 591 | 743 |
| 25 | 72 | 379 |
| 26 | 38 | 119 |
| 27 | 386 | 834 |
| 28 | 89 | 150 |
| 29 | 28 | 27 |
| 30 | 115 | 71 |
| 31 | 8 | 106 |
| 32 | 12 | 30 |
| 33 | 116 | 131 |
| 34 | 14 | 47 |
| 35 | 122 | >5000 |
| 36 | 55 | 66 |
| 37 | 41 | 254 |
| 38 | 5 | 15 |
| 39 | 0.9 | 7 |
| 40 | 77 | 943 |
| 41 | 11 | 30 |
| 42 | 8 | 24 |
| 43 | 5 | 86 |
| 44 | 3 | 32 |
| 45 | 3 | 36 |
| 46 | >5000 | 476 |
| 47 | <3 | 19 |
| 48 | 2 | 7 |
| 49 | 1 | 9 |
| 50 | 6 | 75 |
| 51 | 3 | 24 |
| 52 | 3 | 46 |
| 53 | 3 | 141 |
| 54 | 29 | 17 |
| 55 | 72 | 181 |
| 56 | 2 | 23 |
| 57 | 9 | 27 |
| 58 | 201 | 16 |
| 59 | 504 | 255 |
| 60 | 156 | 233 |
| 61 | 4 | 41 |
| 62 | 292 | 565 |
| 63 | 55 | 66 |
| 64 | 9 | 24 |
| 65 | 3 | 10 |
| 66 | 6 | 42 |

### Example 12

Intraperitoneal Delayed Type Hypersensitivity (IP-DTH) Response. A Method for Analyzing Effects of Integrin Antagonists In Vivo

Background: Integrin antagonists are meant to interfere with the binding or adhesion of immune cells, such as lymphocytes, monocytes and eosinophils that bear integrin receptors to counter-receptors that exist on endothelial cells in the vasculature. Among those integrin-bearing cells, cells that are positive for α4β7 integrin are found in the mesenteric system and in the gut, and would comprise many of the cells recruited to a peritoneal antigen challenge. One can maximize the number of α4β7 integrin-positive cells recruited by inducing an intraperitoneal delayed type hypersensitivity response to antigen that will recruite antigen-responsive cells from the mesenteric lymph nodes. An inhibitor of α4β7 integrin should prevent the recruitment of these cells to the site of antigen challenge. Since α4β7 integrin-positive cells are considered to be gut-homing, and are found in greater abundance in inflamed tissues of the GI tract and pancreas, preventing recruitment of α4β7 integrin-positive cells in an IP-DTH model might predict efficacy in inflammatory diseases of the GI tract or pancrease. Those diseases might include Crohn's disease and ulcerative colitis, as well as other forms of colitis or inflammatory bowel disease, and pancreatitis.

The antigenic challenge will induce a delayed type hypersensitivity response. In this model, animals are primed with antigen, then 7 days later are challenged intraperitoneally with the same antigen. During the ensuing 24-48 hours, cells that have been primed to recognize this antigen will be recruited to the challenge site. If the site is the peritoneal cavity, the recruited cells can be obtained by lavaging the cavity with a physiological buffer and collecting the lavage fluid.

The contribution of α4β7 integrin positive cells to the peritoneal cavity cell population can be ascertained by using flow cytometry to evaluate their relative percent in this population.

Method:

Mice were primed, via intra-peritoneal administration, with 25 micrograms ovalbumin in a physiological buffer that may or may not contain alum as an adjuvant. 7 days later, the mice were challenged with 25 micrograms ovalbumin via intra-peritoneal administration. Compounds were administered either orally (po), or subcutaneously (sc), either once daily or twice daily, for 2 days, starting on the day of antigen challenge.

Forty-eight hours after antigen challenge, the elicited cells were harvested from the peritoneal cavity by lavaging the cavity in physiological saline or phosphate buffered saline, with calcium and magnesium salts. The cells obtained were washed in Staining Buffer consisting of phosphate buffered saline, 1% bovine serum albumin and 0.1% sodium azide, and resuspended at a concentration of 2×10⁷ cells/ml. 1×10⁶ cells were placed into a 96-well V-bottom plate for staining.

The cells were stained with fluorochrome-coupled antibody to α4β7 integrin or a primary antibody to α4β7 integrin followed by a secondary fluorochrome-coupled antibody. Each staining step was carried out at 4° C. for 30 to 45 minutes with gentle shaking, and was followed by 4 washes with Staining Buffer at 4° C. Finally the cells were resuspended in 200 microliters of 1% paraformaldehyde in phosphate buffered saline. The cells were then transferred to test tubes and maintained at 4° C. until analyzed by flow cytometry to determine numbers of α4β7-postive cells. Flow cytometry was perfomed with a Becton-Dickenson FACSort (B-D instruments).

Comparisons were made between numbers of α4β7-positive cells in samples taken from antigen-treated animals and numbers of α4β7-positive cells taken from antigen-treated animals administered experimental compounds.

| | | | | Percent |
|---|---|---|---|---|
| | | | | Decrease in |
| | Dose | Route of | Dose | recruited |
| Compound No. | (mg/kg) | Admin | Regimen | α4β7+ cells |
| 16 | 80 | sc | qd × 2 | 56 |
| 3 | 80 | sc | qd × 2 | 20 |

### Example 13

### Leukocytosis Procedure

Background: Leukocytosis is the increase in circulating white blood cells (leukocytes). Luekocytosis can be caused by preventing leukocyte binding to integrin counter-receptor adhesion molecules expressed on high endothelial venules. This cell adhesion occurs between immunoglobulin superfamily molecules and integrins. Relevant examples of these paired interactions include Intracellular Adhesion Molecule-1 and αLβ2 integrin, Vascular Cell Adhesion Molecule-1 and α4β1 integrin, and Mucosal Addressin Cell Adhesion Molecule-1 and α4β7 integrin, respectively.

In this model, a compound that antagonizes these leukocyte-endothelial interactions will cause an increase in circulating leukocytes, defined as leukocytosis, as measured at 1-1.5 hours post-administration. This leukocytosis is indicative that normal lymphocyte or leukocyte emigration from the peripheral circulation was prevented. Similar emigration of cells out of the circulation into inflamed tissues is responsible for the progression and maintenance of the inflammatory state. Leukocytosis is an indication that lymphocyte and leukocyte extravasation is prevented, and is predictive of general anti-inflammatory activity.

Methodology

1 week prior to being tested, 7-10 week old female Balb/c mice, n=8 per group, were bled and randomized according to leukocyte counts. One week later, the mice were administered a test compound orally or subcutaneously and then bled 1-1.5 hours after drug administration, approximately one hour after the peak blood concentration of the compound had occurred. Whole blood, 250-350 microliters, was collected from each mouse into potassium-EDTA serum collection tubes (Becton-Dickenson) and mixed to prevent clotting.

Cell counts and differential counts on the whole blood preparation were performed using an Advia 120 Hematology System (Bayer Diagnostics). Cell counts as total leukocytes and as total lympohcytes were made and compared to counts made from mice dosed with vehicle only. Data were reported as percent of vehicle control for lymphocyte counts and total leukocyte counts. Statistical analyses were performed using ANOVA with Dunnet's multiple comparison test

| | | | | Lymphocyte Counts | | | Total Leukocyte Counts | | |
|---|---|---|---|---|---|---|---|---|---|
| Compound | | | | % of Vehicle Control | | | % of Vehicle Control | | |
| No. | α4β1 | α4β7 | Route | 3 mg/kg | 10 mg/kg | 30 mg/kg | 3 mg/kg | 10 mg/kg | 30 mg/kg |
| 39 | 0.9 | 7 | sc | 205.2 | 150.3 | 253.9 | 175.1 | 134.3 | 212.2 |
| | | | | p < 0.05 | | p < 0.05 | p < 0.05 | | p < 0.05 |

### Example 14

### Phorbol 12-Myristate 13-Acetate (PMA)-Induced Inflammation in Mouse Ear Skin and Measurement of Eosinophil Peroxidase

Phorbol 12-myristate 13-acetate (PMA) when applied to skin generates a vigorous recruitment of immune cells to the site of application. Over a 24 hour period, there is accumulation of fluid and cells to the inflamed site, which is an general indicator of an imflammatory response. Among the recruited cells are eosinophils and neutrophils. Eosinophils can migrate into an inflamed or infected tissue via α4β1 integrin interactions with vascular cell adhesion molecule-1 (VCAM-1) counter-receptors on vascular endothelial cells, and via α4β7 integrin to mucosal addressin cellular adhesion molecule-1 (MAdcAM-1) on vascular endothelial cells in the gastrointestinal tract and mesenteric system. The recruited esoinophils can be quantified by measuring the presence of eosinophil peroxidase in a sample of the homogenized tissue. Those that are recruited to the inflamed site in the ear do so via integrin-Ig superfamily receptor pairs that notably include α4β1 integrin—VCAM-1 interactions.

### Induction of Ear Inflammation and Treatment of Animals

Female BALB/C mice were ordered at 6 weeks of age and 16-18 grams from Charles River were used between 6-10 weeks of age. The animals were randomly assigned to groups of 10 (5/box) and housed in groups in plastic cages in a room with 12 h light-dark cycle and controlled temperature and humidity. They received food and water ad libitum.

Phorbol 12-myristate 13-acetate (PMA) used in the experiment was dissolved as 5 mg per ml stock in dimethyl sulfoxide (DMSO) and stored frozen as 20 microliter aliquots. For application to mouse ears, each aliquot was diluted in 2 ml with acetone. The right ear of each mouse was treated topically with 20 microliters of acetone solution (10 microliters to each side of the ear) containing either 1 microgram of phorbol 12-myristate 13-acetate (PMA) or acetone alone. Drugs that were tested; were administered orally at −1 and +3 hours relative to PMA application. In one experiment, various intraperitoneal dosing regimens were explored (dosing once at 0 hr, dosing twice at 0 and +4 hr, or dosing three times at 0, +4 and +8 hours relative to time of PMA application.

### Estimation of Ear Tissue Eosinophil Content BY Assay of Eosinophil Peroxidase

Mice were sacrificed 24 hrs after PMA application. The right ear was punched with a 6 mm tissue punch and the tissue was placed in a tube on dry ice and kept frozen until extraction.

Materials and Supplies:

0.5% hexadecyltrimethylammonium bromide (HTAB) (Sigma-Aldrich, no. H 5882) w/v in 0.1 M sodium acetate and 0.1 M sodium sulfate buffer, pH 6.0.

Phosphate citrate buffer with urea hydrogen peroxide (Sigma-Aldrich, no. P9305)

o-phenylenediaminedihydrochloride (Sigma-Aldrich, no. P1063).

4N H₂SO₄

96-well flat bottom polystyrene tissue culture plate (Costar, no. 3595) 2 mL polypropylene conical microcentrifuge tubes.

Brinkman Polytron—large head.

Methods

### Substrate Buffer Preparation

The substrate buffer was prepared by dissolving one tablet of phosphate citrate buffer with urea hydrogen peroxide in 100 ml of water in which one tablet containing 60 mg of o-phenylenediaminedihydrochloride was added.

### Eosinophil Peroxidase Extraction

Ear tissue samples were homogenized in 2 ml of HTAB for 15 seconds at speed 5.5 with a Polytron (large head) (Brinkman Instruments). The homogenate was stored at

−20° C. until assayed.

### Eosinophil Peroxidase Assay

On the day of eosinophil peroxidase measurements, the ear tissue homogenates were heated to 60° C. for 2 hrs in a water bath to guarantee the maximal recovery of eosinophil peroxidase activity. After heating, samples were transferred into a 2 ml conical polypropylene microcentrifuge tube and spun for 10 minutes at 10,000×g in a microcentrifuge to clear debris. Samples were typically tested at either a 1:2 or 1:4 dilution made with HTAB. 100 microliters of sample was pipetted into a 96-well microtiter plate (Costar no. 3595) followed by addition of 100 microliters of substrate buffer. After 10 minutes of incubation at room temperature the reaction was stopped by adding 50 microliters of 4N H₂SO₄. Absorbance was read at 490 nm for the specific with subtraction of a 650 nm noise signal using a Thermomax 96-well spectrophotmetric plate reader (Molecular Devices). Analysis was performed using ANOVA on EXCEL and determining significance with Dunnett's Significant Difference compared to normal controls who received only an acetone application to the ear.

| | | | | | | P-value vs. |
|---|---|---|---|---|---|---|
| | | | | | | Vehicle |
| Compound | | | | % Inhibition | | Treated |
| Number or | Total | | Dosing | Eosinophil | | PMA |
| Name | Dose | Dosing Route | Regimen | Peroxidase | SE | Control |
| Dexamethasone | 5 | ip | qd | 89.7 | 5.3 | p < 0.05 |
| 40 | 40 | po | bid | −36.5 | 27.1 | ns |
| 32 | 40 | po | bid | 7.8 | 8.6 | ns |
| 41 | 40 | po | bid | 14.8 | 13.8 | ns |
| 37 | 40 | po | bid | 21.3 | 7.1 | ns |
| 16 | 40 | po | bid | 4.8 | 16.3 | ns |
| 53 | 40 | po | bid | 56.6 | 13.1 | P < 0.05 |
| 4 | 40 | po | bid | 38.4 | 13.6 | ns |
| Dexamethasone | 5 | po | bid | 109.0 | 2.8 | P < 0.05 |
| 53 | 40 | po | bid | 27.3 | 6.7 | ns |
| 53 | 10 | po | bid | 13.7 | 4.3 | ns |
| Dexamethasone | 5 | po | bid | 83.6 | 4.0 | p < 0.05 |

ns = non-significant
p < 0.05 = statistically significant

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound of Formula (I): 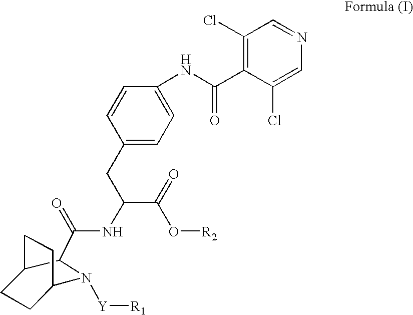
wherein
Y is selected from the group consisting of a bond, —C(O)—, —C(O)O— and —C(O)NH—;
R₁ is selected from the group consisting of R₃ and R₄;
R₂ is selected from the group consisting of hydrogen and C₁₋₄alkyl; wherein C₁₋₄alkyl is optionally substituted with one to three substituents independently selected from amino, N—(C₁₋₄alkyl)amino, N,N—(C₁₋₄dialkyl)amino, hydroxy, C₁₋₄alkoxy, —CF₃ and —OCF₃;
R₃ and R₅ are independently selected from the group consisting of cycloalkyl, heterocyclyl, aryl and heteroaryl optionally substituted with one to five substituents independently selected from the group consisting of halogen, C₁₋₆alkyl, C₂₋₆alkenyl, C₂₋₆alkynyl, C₁₋₆alkoxy, C₁₋₆alkylcarbonyl, C₁₋₈alkoxycarbonyl, carboxyl, aryl, heteroaryl, arylcarbonyl, heteroarylcarbonyl, arylsulfonyl, amino, N—(C₁₋₆alkyl)amino, N,N—(C₁₋₆dialkyl)amino, —CF₃ and —OCF₃; and, wherein the heterocycl, aryl and heteroaryl substituents and the aryl portion of the arylcarbonyl substituent are optionally substituted with one to five substituents independently selected from the group consisting of halogen, C₁₋₈alkyl, C₂₋₆alkenyl, C₂₋₆alkynyl, C₁₋₆alkoxy, carboxyl, amino, N—(C₁₋₆alkyl)amino, N,N—(C₁₋₆dialkyl)amino, —CF₃ and —OCF₃;
R₄, is independently selected from the group consisting of C₁₋₆alkyl, C₂₋₆alkenyl, C₂₋₆alkynyl, and (halo)₁₋₃(C₁₋₆)alkyl; wherein C₁₋₆alkyl, C₂₋₆alkenyl and C₂₋₆alkynyl are optionally substituted on a terminal carbon with one to three substituents independently selected from R₅;
provided that when Y is C(O)NH and R₄ is a C₁₋₆alkyl substituted with R₅ and;
provided that when Y is a bond then R₁ is R₄ and R₄ is C₁₋₆alkyl optionally substituted with R₅, and R₅ is selected from the group consisting of heterocyclyl and aryl optionally substituted with halo, C₁₋₄alkyl, C₁₋₄alkoxy, N—(C₁₋₄alkyl)amino, N,N—(C₁₋₄dialkyl)amino, —CF₃ and —OCF₃
and pharmaceutically acceptable salts, racemic mixtures, diastereomers and enantiomers thereof.

2. The compound of claim 1 wherein Y is selected from the group consisting of —C(O)— and —C(O)O—

3. The compound of claim 1 wherein Y is selected from —C(O)—.

4. The compound of claim 1 wherein R₂ is selected from the group consisting of hydrogen and C₁₋₄alkyl.

5. The compound of claim 1 wherein R₂ is selected from the group consisting of hydrogen and methyl.

6. The compound of claim 1 wherein R₁ is R₃.

7. The compound of claim 1 wherein R₃ is selected from the group consisting of cycloalkyl, aryl and heteroaryl optionally substituted with one to five substituents independently selected from the group consisting of halogen, C₁₋₆alkyl, C₂₋₆alkenyl, C₂₋₆alkynyl, C₁₋₆alkoxy, C₁₋₈alkylcarbonyl, C₁₋₆alkoxycarbonyl, carboxyl, aryl, heteroaryl, arylcarbonyl, heteroarylcarbonyl, arylsulfonyl, amino, N—(C₁₋₆alkyl)amino, N,N—(C₁₋₆dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₆alkyl, C₂₋₆alkenyl, C₂₋₆alkynyl, C₁₋₆alkoxy, carboxyl, amino, N—(C₁₋₆alkyl)amino, N,N—(C₁₋₆dialkyl)amino, —CF₃ and —OCF₃.

8. The compound of claim 1 wherein R₃ is selected from the group consisting of aryl optionally substituted with one to five substituents independently selected from the group consisting of halogen, C₁₋₆alkyl, C₂₋₆alkenyl, C₂₋₆alkynyl, C₁₋₆alkoxy, C₁₋₆alkylcarbonyl, C₁₋₆alkoxycarbonyl, carboxyl, aryl, heteroaryl, arylcarbonyl, heteroarylcarbonyl, arylsulfonyl, amino, N—(C₁₋₈alkyl)amino, N,N—(C₁₋₈dialkyl)amino, —CF₃ and —OCF₃; and, wherein the aryl and heteroaryl substituents and the aryl portion of the arylcarbonyl substituent are optionally substituted with one to five substituents independently selected from the group consisting of halogen, C₁₋₆alkyl, C₂₋₆alkenyl, C₂₋₆alkynyl, C₁₋₆alkoxy, carboxyl, amino, N—(C₁₋₆alkyl)amino, N,N—(C₁₋₆dialkyl)amino, —CF₃ and —OCF₃.

9. The compound of claim 1 wherein Y is a bond and R₄ is C₁₋₄alkyl optionally substituted with R₅, and R₅ is selected from the group consisting of heterocyclyl and aryl optionally substituted with C₁₋₄alkyl, C₁₋₄alkoxy, N—(C₁₋₄alkyl)amino, N,N—(C₁₋₄dialkyl)amino, —CF₃ and —OCF₃.

10. The compound of claim 1 wherein Y is —C(O)NH— and R₄ is methyl and R₅ phenyl and R₂ is hydrogen.

11. The compound of claim 1 wherein Y is —C(O)NH—, R₃ is selected from the group consisting of phenyl, 4-tolyl, 2-chlorophenyl, 3-chlorophenyl and R₂ is hydrogen.

12. The compound of claim 1 having the Formula (I): 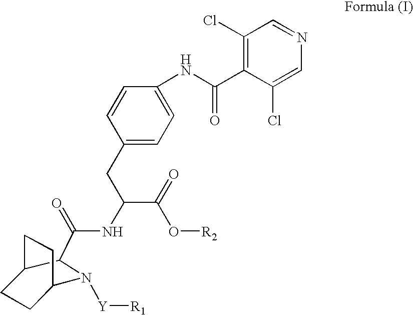
wherein
Y is selected from the group consisting of a —C(O)— and —C(O)O—:
R₁ is selected from the group consisting of R₃ and R₄;
R₂ is selected from the group consisting of hydrogen and C₁₋₄alkyl;
R₃ is aryl optionally substituted with one to three substituents independently selected from the group consisting of halogen, methyl, methoxy, carboxyl, aryl, amino, N-methylamino, N,N-dimethylamino, —CF₃ and —OCF₃; wherein the aryl substituent is optionally substituted with one to three substituents sleclected from the group consisting of halogen, methyl, methoxy, carboxyl, amino, N-methylamino, N,N-dimethylamino, —CF₃ and —OCF₃;
R₄, is selected from the group consisting of C₁₋₆alkyl, C₂₋₆alkenyl, C₂₋₆alkynyl, and (halo)₁₋₃(C₁₋₆)alkyl; wherein C₁₋₆alkyl, C₂₋₆alkenyl and C₂₋₆alkynyl are optionally substituted on a terminal carbon with one substituent independently selected from R₁₄;
R₅ is independently selected from the group consisting of cycloalkyl, heterocyclyl, aryl and heteroaryl optionally substituted with one to five substituents independently selected from the group consisting of halogen, C₁₋₆alkyl, C₁₋₆alkoxy, C₁₋₆alkylcarbonyl, C₁₋₆alkoxycarbonyl, carboxyl, amino, N—(C₁₋₈alkyl)amino, N,N—(C₁₋₈dialkyl)amino, —CF₃ and —OCF₃;
and pharmaceutically acceptable salts, racemic mixtures, diastereomers and enantiomers thereof.

13. The compound of claim 12 wherein R₃ is selected from the group consisting of 2,5-dimethoxyphenyl, 2-fluorophenyl, 3,5-dichlorophenyl and 4-fluoro-biphenyl-2-yl.

14. The compound of claim 12 wherein R₄ is methyl and R₅ is selected from the group consisting of phenyl; 4-methylphenyl; 2-methoxyphenyl; 3-methoxyphenyl; 4-methoxyphenyl; 3,5-dimethoxyphenyl; 3,6-dimethoxyphenyl; 2,6-dichlorophenyl; 2-trifluoromethylphenyl; naphthalene-2-yl; thiophen-2-yl; thiophen-3-yl; pyridin-2-yl; pyridin-3-yl; pyridin-4-yl; 5-methyl-pyrazol-1-yl; tetrazol-1-; benzo[1,3]dioxol-5-yl; benzo[b]thiophen-3-yl; and tetrahydropyran-4-yl.

15. The compound of claim 12 wherein R₄ is ethyl and R₅ is selected from the group consisting of phenyl; 3-methoxyphenyl; 4-methoxyphenyl; 4-chlorophenyl; 2-fluorophenyl; 4-trifluoromethylphenyl; 3,5-ditrifluoromethylphenyl; thiophen-2-yl; 2-piperazin-1-yl; 2(4-tert-butoxycarbonyl-piperazin-1-yl); 2-piperidin-1-yl; fyran-3-yl; and 2-cyclopentyl.

16. The compound of claim 12 wherein R₄ is vinyl and R₅ is selected from the group consisting of 1-methyl-2-phenyl; and 2-(2-methoxyphenyl).

17. The compound of claim 12 wherein R₄ is propyl and R₅ is selected from the group consisting of -phenyl; 3-cyclohexyl; and 2,2-dimethyl.

18. The compound of claim 12 wherein R₄ is butyl and R₅ is selected from the group consisting of 3 3,3-dimethyl; and 3-methyl.

19. The compound of claim 1 having Formula (I): 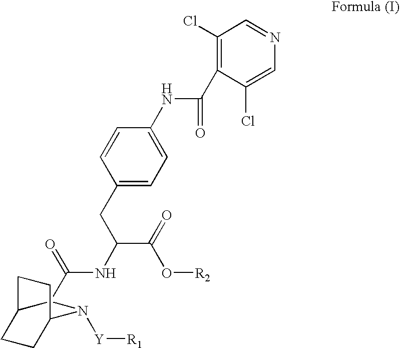
wherein R₂ is hydrogen and Y and R₁ are dependently selected from the group consisting of:

| Y | R₁ | * Config. |
|---|---|---|
| —C(O)— | 3-Methoxy-benzyl | R, S |
| —C(O)— | 2-Trifluoromethyl- | S |
| | benzyl | |
| —C(O)— | Thiophen-3-ylmethyl | R, S |
| —C(O)— | Pyridin-2-ylmethyl | R, S |
| —C(O)— | Pyridin-3-ylmethyl | R, S |
| —C(O)— | 2,6-Dichloro-benzyl | R, S |
| —C(O)— | Naphthalen-2-ylmethyl | R, S |
| —C(O)— | 2,5-Dimethoxyphenyl | R, S |
| —C(O)— | 2-Fluorophenyl | R, S |
| —C(O)— | 3,5-Dichlorophenyl | R, S |
| —C(O)— | 4-Fluoro-biphenyl-2-yl | R, S |
| —C(O)— | 2- | R, S |
| | Trifluoromethylphenyl | |
| bond | 3,3-Dimethyl-butyl | S |
| bond | Benzyl | S |
| bond | 5-Chlorothiophe-2- | S |
| | ylmethyl | |
| —C(O)NH— | Phenyl | S |
| —C(O)NH— | 4-Tolyl | S |
| —C(O)NH— | 2-Chlorophenyl | S |
| —C(O)NH— | 3-Chlorophenyl | S |
| —C(O)NH— | 4-Chlorophenyl | S |
| —C(O)NH— | Benzyl | S |
| —C(O)— | 5-Methyl-pyrazol-1- | R, S |
| | ylmethyl | |
| —C(O)— | 4-Methyl-benzyl | S |
| —C(O)— | tert-Butyl | R, S |
| —C(O)— | 2,2-Dimethyl-propyl | R, S |
| —C(O)— | 2-Cyclopentyl-ethyl | R, S |
| —C(O)— | 2-Methoxy-benzyl | R, S |
| —C(O)— | 4-Methoxy-benzyl | R, S |
| —C(O)— | 3,5-Dimethoxy-benzyl | |
| —C(O)— | Thiophen-2-ylmethyl | R, S |
| —C(O)— | 3,6-Dimethoxy-benzyl | |
| —C(O)— | 2,2-Dimethyl-propyl | S |
| —C(O)— | 2-(4-tert- | S |
| | Butoxycarbonyl- | |
| | piperazin-1-yl)-ethyl | |
| —C(O)— | Benzo[b]thiophen-3- | R, S |
| | ylmethyl | |
| —C(O)— | 3-Cyclohexyl-propyl | R, S |
| —C(O)— | 3-Methyl-butyl | R, S |
| —C(O)— | Phenethyl | R, S |
| —C(O)— | 1-Methyl-2-phenyl-vinyl | S |
| —C(O)— | Tetrahydro-pyran-4- | S |
| | ylmethyl | |
| —C(O)—O— | Benzyl | S |
| —C(O)— | 2-Piperidin-1-yl-ethyl | R, S |
| —C(O)— | Pyridin-4-ylmethyl | R, S |
| —C(O)— | 2-(2-Methoxy-phenyl)- | S |
| | vinyl | |
| —C(O)— | 2-Benzo[1,3]dioxol-5- | R, S |
| | yl-vinyl | |
| —C(O)— | Thiophen-2-yl-ethyl | R, S |
| —C(O)— | 4-Methoxyphenethyl | R, S |
| —C(O)— | 3-Methoxyphenethyl | R, S |
| —C(O)— | 4- | R, S |
| | Trifluoromethylphenethyl | |
| —C(O)— | 4-Chlorophenethyl | R, S |
| —C(O)— | 2-Fluorophenethyl | R, S |
| —C(O)— | 2-Piperazin-1-yl-ethyl | R, S |
| —C(O)— | Furan-3-yl-ethyl | S |
| —C(O)— | 3,5-Ditrifluoromethyl- | S |
| | phenethyl | |
| —C(O)— | 4-Dimethylamino- | S |
| | benzyl | |
| —C(O)— | Pyridin-3-yl-ethyl | R, S |
| —C(O)— | Phenylethynyl | R, S |

20. The compound of claim 19 wherein Y and R₁ are dependently selected from the group consisting of:

| | | * |
|---|---|---|
| Y | R₁ | Config. |
| —C(O)— | 3-Methoxy-benzyl | R, S |
| —C(O)— | Thiophen-3-ylmethyl | R, S |
| —C(O)— | Pyridin-2-ylmethyl | R, S |
| —C(O)— | Pyridin-3-ylmethyl | R, S |
| —C(O)— | Naphthalen-2-ylmethyl | R, S |
| —C(O)— | 4-Fluoro-biphenyl-2-yl | R, S |
| —C(O)— | 2- | R, S |
| | Trifluoromethylphenyl | |
| —C(O)— | 5-Methyl-pyrazol-1- | R, S |
| | ylmethyl | |
| —C(O)— | 4-Methyl-benzyl | S |
| —C(O)— | tert-Butyl | R, S |
| —C(O)— | 2,2-Dimethyl-propyl | R, S |
| —C(O)— | 2-Cyclopentyl-ethyl | R, S |
| —C(O)— | Benzyl | S |
| O— | | |
| —C(O)— | 2-Methoxy-benzyl | R, S |
| —C(O)— | 4-Methoxy-benzyl | R, S |
| —C(O)— | 3,5-Dimethoxy-benzyl | |
| —C(O)— | Thiophen-2-ylmethyl | R, S |
| —C(O)— | 2,2-Dimethyl-propyl | S |
| —C(O)— | Benzo[b]thiophen-3- | R, S |
| | ylmethyl | |
| —C(O)— | 3-Cyclohexyl-propyl | R, S |
| —C(O)— | 3-Methyl-butyl | R, S |
| —C(O)— | Phenethyl | R, S |
| —C(O)— | 1-Methyl-2-phenyl-vinyl | S |
| —C(O)— | Tetrahydro-pyran-4- | S |
| | ylmethyl | |
| —C(O)— | Benzyl | S |
| O— | | |
| —C(O)— | 2-Piperidin-1-yl-ethyl | R, S |
| —C(O)— | Pyridin-4-ylmethyl | R, S |
| —C(O)— | 2-(2-Methoxy-phenyl)- | S |
| | vinyl | |
| —C(O)— | 2-Benzo[1,3]dioxol-5- | R, S |
| | yl-vinyl | |
| —C(O)— | Thiophen-2-yl-ethyl | R, S |
| —C(O)— | 4-Methoxyphenethyl | R, S |
| —C(O)— | 3-Methoxyphenethyl | R, S |
| —C(O)— | 4- | R, S |
| | Trifluoromethylphenethyl | |
| —C(O)— | 4-Chlorophenethyl | R, S |
| —C(O)— | 2-Fluorophenethyl | R, S |
| —C(O)— | 2-Piperazin-1-yl-ethyl | R, S |
| —C(O)— | Furan-3-yl-ethyl | S |
| —C(O)— | 3,5-Ditrifluoromethyl- | S |
| | phenethyl | |
| —C(O)— | 4-Dimethylamino- | S |
| | benzyl | |
| —C(O)— | Pyridin-3-yl-ethyl | R, S |
| —C(O)— | Phenylethynyl | R, S |

21. A pharmaceutical composition comprising a compound of claim 1 and a pharmaceutically acceptable carrier.

22. A method for the treatment of an integrin mediated disorder ameliorated by inhibition of an α4 integrin receptor comprising administering to a subject in need thereof a therapeutically effective amount of a compound of claim 1.

23. The method of claim 22 wherein the α4 integrin receptor is selected from the group consisting of the α4β1 and α4β7 integrin receptor.

24. The method of claim 22 wherein the integrin mediated disorder is selected from the group consisting of inflammatory disorders, autoimmune disorders and cell-proliferative disorders.

25. The method of claim 22 wherein the integrin mediated disorder is selected from the group consisting of inflammation disorders, autoimmunity disorders, asthma, bronchoconstriction, restenosis, atherosclerosis, psoriasis, rheumatoid arthritis, inflammatory bowel disease, irritable bowel disease, irritable bowel syndrome, transplant rejection and multiple sclerosis.

26. The compound of claim 22 wherein the integrin mediated disorder is selected from the group consisting of asthma, bronchoconstriction, restenosis, atherosclerosis, psoriasis, rheumatoid arthritis, inflammatory bowel disease, irritable bowel disease, irritable bowel syndrome, transplant rejection and multiple sclerosis.

27. The compound of claim 26 wherein the integrin mediated disorder is selected from the group consisting of asthma, bronchoconstriction, restenosis, atherosclerosis, irritable bowel syndrome and multiple sclerosis.

28. The method of claim 22 wherein the therapeutically effective amount of the compound of claim 1 is from about 0.01 mg/kg/day to about 300 mg/kg/day.
